(19) United States
(12) Patent Application Publication
(10) Pub. No.: US 2019/0257816 A1
(43) Pub. Date: Aug. 22, 2019

(54) DIFFERENTIAL BH3 MITOCHONDRIAL PROFILING

(71) Applicant: Eutropics Pharmaceuticals, Inc., Cambridge, MA (US)

(72) Inventor: Michael H. CARDONE, Dorchester, MA (US)

(21) Appl. No.: 15/698,281

(22) Filed: Sep. 7, 2017

Related U.S. Application Data
(63) Continuation of application No. 14/905,519, filed on Jan. 15, 2016, now abandoned.
(60) Provisional application No. 61/847,750, filed on Jul. 18, 2013.

(51) Int. Cl. G01N 33/50; A61K 49/00

(52) U.S. Cl. CPC G01N 33/5011; G01N 33/5079; G01N 2800/52; G01N 2333/47; A61K 49/0008

(57) ABSTRACT

The present invention relates to methods of determining cancer cell sensitivity to treatment by correlating the pattern of sensitivity of the cell to a panel of BH3 domain peptides. The invention also provides a method applying an algorithm to said pattern to predict therapeutic efficacy and of monitoring the shift in cell sensitivity to a therapeutic during treatment.

### PRIORITY

This application claims the benefit of U.S. Provisional Application No. 61/847,750 filed Jul. 18, 2013 which is hereby incorporated by reference herein in its entirety.

### FIELD OF THE INVENTION

The present invention relates to methods that are useful in evaluating tumors in human samples.

### BACKGROUND

The use of predictive and prognostic biomarkers paired with targeted cancer therapies may hold the key to reducing drug development time, improving drug efficacy, and guiding clinical decision making. While there are advances in cancer treatment, chemotherapy remains largely inefficient and ineffective. One reason for the generally poor performance of chemotherapy is that the selected treatment is often not closely matched to the individual patient's disease. A personalized medicine approach that couples precision diagnostics with therapeutics, especially targeted therapeutics, is considered a highly promising method for enhancement of the effectiveness of current and future drugs. Biomarkers can facilitate the development and use of such targeted therapeutics as well as standard of care therapies.

To date there are only a handful of biomarkers that have added value to clinical oncology practice. In part this is because perceived markers often are correlative but not causal to drug mechanism. Even when the “biomarker” biology does line up with the pharmacology of the companion therapy there is still significant challenge to predicting how a drug will work in a patient. Beyond this, the path to clinical development requires the participation of physician-scientists who see the value of the test and believe it can bring benefit to their patients.

Mitochondrial profiling (AKA BH3 profiling) measures the functionality of a pivotal causal factor to cancer cell response to chemotherapy. Specifically, mitochondrial profiling measures the functionality of proteins at the surface of the mitochondria that control apoptosis. Many chemotherapies rely on apoptosis to be effective. The readout of the test provides a response of the mitochondria to BH3 domains of the pre-apoptotic BH3 only proteins, which has previously been used to provide a general sense of chemosensitivity or chemoresponsiveness to therapies.

### SUMMARY OF THE INVENTION

The present invention provides a method of differential mitochondrial profiling to determine a cancer cell's predisposition to undergo apoptosis. Mitochondria that are predisposed to apoptosis are dependent on anti-apoptotic protein function to sequester pro-apoptotic Bcl-2 family proteins, and in doing so prevent mitochondrial outer membrane permeabilization (MOMP). Exposure to ligands comprised of BH3 peptides or functionally similar small molecules (i.e. BH3 mimetics) releases activating pro-apoptotic proteins from being sequestered and increases MOMP, a hallmark of apoptosis, which can be measured, for example, by the degree of staining by a mitochondrial dye, or by cytochrome C release. “Mitochondrial priming” is the degree to which the anti-apoptotic Bcl-2 family proteins are bound to pro-apoptotic Bcl-2 family proteins, and the percent of mitochondrial priming indicates the degree to which apoptosis is likely to proceed in response to upstream cues. The percent priming is then correlated to patient response.

The present invention provides a method of exposing cancer cells or specimens to one or more therapeutics and/or one or more BH3 peptides or BH3 mimetics to determine the degree of mitochondrial priming for a given sample. The percent mitochondrial priming can be compared to that of a standard test sample, and to the percent mitochondrial priming observed in the same patient throughout treatment to determine the cancer's sensitivity or resistance to treatment which allows a prediction of the continued efficacy of the treatment. This differential mitochondrial profiling allows monitoring of a patient during treatment to observe any shifts in cancer cell priming that will correlate to sensitivity to a treatment to classify the patient into a treatment/prognosis group, thereby guiding future treatment. The application of an algorithm derived from the read-out from the mitochondrial profile allows unique correlation to particular treatments. Assay ligands that provide an increased range of perturbations of the Bcl-2 family complexes provide better correlation between percent priming and patient response than BH3 containing peptides alone.

In one aspect, the invention provides a method for determining a cancer treatment for a patient, comprising: a) isolating a cancer cell or specimen from said patient; b) contacting said cancer cell or specimen with one or more therapeutic agents and one or more BH3 domain peptides or mimetics thereof; c) comparing the level of mitochondrial priming in a test sample with that of the cancer cell or specimen, and determining whether said BH3 domain peptide or mimetic thereof induces apoptosis in said cancer cell to produce a mitochondrial profile for the patient's tumor or cancer cell specimen; d) determining a correlation between the data obtained from the mitochondrial profile and the sensitivity of said cell or specimen to said treatment; and e) classifying the patient for likelihood of clinical response to one or more cancer treatments, wherein the mitochondrial profile correlates with treatment efficacy.

In one aspect, the invention provides a method for predicting cancer sensitivity to treatment, comprising: a) isolating a cancer cell or specimen from said patient; b) contacting said cancer cell or specimen with one or more therapeutic agents and one or more BH3 domain peptides or mimetics thereof; c) comparing the level mitochondrial priming in a test sample with that of the cancer cell or specimen, and determining whether said BH3 domain peptide or mimetic thereof induces apoptosis in said cancer cell to produce a mitochondrial profile for the patient's tumor or cancer cell specimen; d) determining a correlation between the data obtained from the mitochondrial profile and the sensitivity of said cell or specimen to said treatment; and e) classifying the patient for likelihood of clinical response to one or more cancer treatments, wherein the mitochondrial profile correlates cancer sensitivity to treatment

In one aspect, the invention provides a method for monitoring cancer treatment efficacy for a patient, comprising: a) isolating a cancer cell or specimen from said patient before, during, and/or after treatment; b) contacting said cancer cell or specimen with one or more therapeutic agents and one or more BH3 domain peptides or mimetics thereof; c) comparing the predisposition towards drug induced apoptosis of a cancer cell in a sample by measuring the level of mitochondrial priming using BH3 domain peptides or mimetics thereof; d) comparing the predisposition towards drug induced apoptosis of a cancer cell in a sample from time “0” to that of samples taken at different time points in drug treatment by comparing the level of priming at the different time points; and e) comparing the mitochondrial profiles from the different time points; and f) classifying the patient for likelihood of clinical response to one or more cancer treatments, wherein a change in mitochondrial profile indicates a shift in cell response to treatment.

In one embodiment, apoptosis induction is measured through changes in a marker. In a further embodiment, the marker is a change in mitochondrial membrane potential or cytochrome C release.

In one embodiment, the therapeutic agent is contacted with the cell or specimen in vitro. In another embodiment, the therapeutic agent is contacted with the cell or specimen in vivo. In one embodiment, the cancer treatment is one or more of anti-cancer drugs, chemotherapy, antagonist of an anti-apoptotic protein, surgery, adjuvant therapy, and neoadjuvant therapy. In another embodiment, the cancer treatment is one or more of a BH3 mimetic, proteasome inhibitor, histone deacetylase inhibitor, glucocorticoid, steroid, monoclonal antibody, antibody-drug conjugate, or thalidomide derivative. In another embodiment, the cancer treatment is a BH3 mimetic. In a further embodiment, the BH3 mimetic is selected from the group consisting of EU-5148, ABT-263, and EU-5346. In another embodiment, the cancer treatment is an inhibitor of Bcl-2. In yet another embodiment, the cancer treatment is an inhibitor of Mcl-1.

In one embodiment, the cancer is a hematologic cancer. In further embodiments, the hematologic cancer is selected from acute myelogenous leukemia (AML), multiple myeloma, follicular lymphoma, acute lymphoblastic leukemia (ALL), chronic lymphocytic leukemia, and non-Hodgkin's lymphoma. In one embodiment, the cancer is dependent on BH3 containing polypeptides for survival. In one embodiment, the cancer is dependent on Bcl-2 family polypeptides for survival.

In a further embodiment, the mitochondrial profiling further comprises a) permeabilizing the patient's cancer cells; b) determining a change in mitochondrial membrane potential upon contacting the permeabilized cells with the one or more therapeutics and the one or more BH3 domain peptides or mimetics thereof; and c) correlating a loss of mitochondrial membrane potential with chemosensitivity of the cells to apoptosis-inducing chemotherapeutic agents.

In one embodiment, the mitochondrial profiling comprises use of one or more peptides, wherein the peptide selected from the group consisting of BIM, BIM2A, BAD, BID, HRK, PUMA, NOXA, BMF, BIK, and PUMA2A or variants thereof. In another embodiment, the one or more BH3 domain peptides are selected from the group consisting of SEQ ID NOs: 1-14. In one embodiment, the peptide is used at a concentration of 0.1 μM to 200 μM.

In one embodiment, the specimen is a biopsy selected from a frozen tumor tissue specimen, cultured cells, circulating tumor cells, and a formalin-fixed paraffin-embedded tumor tissue specimen. In a further embodiment, the specimen is a human tumor-derived cell line. In another further embodiment, the specimen is a cancer stem cell. In another embodiment, the specimen is derived from the biopsy of a non-solid tumor. In a further embodiment, the specimen is derived from the biopsy of a patient with multiple myeloma, acute myelogenous leukemia, acute lymphocytic leukemia, chronic lymphogenous leukemia, mantle cell lymphoma, diffuse large B-cell lymphoma, and non-Hodgkin's lymphoma. In another embodiment, the specimen is derived from a circulating tumor cell.

In one embodiment, the method further comprises determining one or more clinical factors of the patient. In a further embodiment, the clinical factor is one or more of age, cytogenetic status, performance, histological subclass, gender, and disease stage.

In one embodiment, the method further comprises predicting a clinical response in the patient.

In one embodiment, the method further comprises comparing the mitochondrial profile of said patient's sample with a test mitochondrial profile of a control, wherein a similarity of said test mitochondrial profile compared to the patient sample mitochondrial profile indicates therapeutic efficacy for said patient.

In one embodiment, the method further comprises applying a biomarker algorithm to the mitochondrial profile activity and correlating the pattern of response with efficacy of treatment.

In one embodiment, the likelihood of clinical response is defined by the following equation:

$$\% Priming = [ 100 * ( \frac{DMSO AUC - {Peptide}_{1} AUC}{DMSO AUC - {CCCP}_{avg} AUC} ) ] {Peptide}_{1} + [ 100 * ( \frac{DMSO AUC - {Peptid}_{2} AUC}{DMSO AUC - {CCCP}_{avg} AUC} ) ] {Peptide}_{2} + … \text{/} ( n peptides )$$

wherein: the AUC comprises either area under the curve or signal intensity; the DMSO comprises the baseline negative control; and the CCCP (Carbonyl cyanide m-chlorophenyl hydrazone) comprises an effector of protein synthesis by serving as uncoupling agent of the proton gradient established during the normal activity of electron carriers in the electron transport chain in the mitochondria comprises the baseline positive control.

In one embodiment, the method further comprises performing the determination before, during, and/or after treatment to determine changes in the mitochondrial profile in a patient, wherein the changes in mitochondrial profiling predict a shift in cell response to treatment. In a further embodiment, the predicted shift in cell response is used to alter patient treatment.

In one embodiment, the cancer is AML and/or MM and the clinical factor is an age profile and/or cytogenetic status.

In one embodiment, said cell or specimen is permeabilized prior to contacting with said one or more therapeutics and said one or more BH3 domain peptides or mimetics thereof. In a further embodiment, the method further comprises contacting said permeabilized cell with a potentiometric dye.

In a further embodiment, the potentiometric dye is JC-1 or dihydrorhodamine 123. In one embodiment, apoptosis is measured by detecting a change in emission of said potentiometric dye.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE FIGURES

FIG. 1 shows representative mitochondrial profiling data in plate format. The figure shows changes in mitochondrial outer membrane permeabilization (MOMP) in response to BH3 peptides are measured in whole-semi-permeabilized cells. The readout is the fluorescent potentiometric dye JC-1.

FIG. 2 shows the work flow for differential mitochondrial profiling. The difference between the profiles at different treatment times is used to assess on target activity and likelihood of further response to treatment.

FIG. 3 shows the mitochondrial response, MOMP, after exposure to BH3 peptide. The mitochondrial profiles of cells that are Mcl-1 primes (NCI-H), Bcl-2 primed (DHL-6), or unprimed (DHL-10) are indicated as a percentage of the positive signal, Bim peptide, or FCCP in Bax, Bak deficient cells. This unprimed pattern is also seen in cells with functional Bax/Bak.

FIG. 4A shows the extent of cell killing observed correlates with the degree of Mcl-1 priming of that cell line as determined by mitochondrial profiling. EU-5148 has comparable activity (48 hours) to MLN9708 in many of the NSLC cancer cell lines treated.

FIG. 4B shows the extent of MOMP in response to Mcl-1 BH3 mimetic EU5149 observed correlates with the degree of Mcl-1 priming of that cell line as determined by mitochondrial profiling. Cells were prepared for the Praedicare Dx assay and the EU-5148 compound was used as the analyte. The readout is the shift in JC1 signal after 90 minutes.

FIG. 5 shows mean tumor burden reduction was observed after treatment with EU-5148, Velcade, or a combination of the two compared with vehicle-only treatment.

FIG. 6 shows the patient response to Velcade combination treatment as predicted by mitochondrial profiling. CD138+ cells were collected from bone marrow before treatment. The response to PUMA peptide was measured as an indication of a “primed state”. The difference in measurement of pre- and post-treatment M protein is used as the patient response criterion.

FIG. 7 shows differential induction of MOMP by different concentrations of Bcl-2/Bcl-xL selective BH3 mimetics, Compound A, Compound B, and ABT263.

FIG. 8 consists of two figures, FIG. 8A and FIG. 8B. FIG. 8A and FIG. 8B show differential induction of MOMP by different concentrations of Mcl-1 selective BH3 mimetic EU5346 (FIG. 8A) and Mcl-1/Bcl-xL selective compound EU5148 (FIG. 8B).

### DETAILED DESCRIPTION OF THE INVENTION

It should be understood that singular forms such as “a,” “an,” and “the” are used throughout this application for convenience, however, except where context or an explicit statement indicates otherwise, the singular forms are intended to include the plural. Further, it should be understood that every journal article, patent, patent application, publication, and the like that is mentioned herein is hereby incorporated by reference in its entirety and for all purposes. All numerical ranges should be understood to include each and every numerical point within the numerical range, and should be interpreted as reciting each and every numerical point individually. The endpoints of all ranges directed to the same component or property are inclusive, and intended to be independently combinable.

“About” includes all values having substantially the same effect, or providing substantially the same result, as the reference value. Thus, the range encompassed by the term “about” will vary depending on context in which the term is used, for instance the parameter that the reference value is associated with. Thus, depending on context, “about” can mean, for example, ±15%, ±10%, ±5%, ±4%, ±3%, ±2%, ±1%, or ±less than 1%. Importantly, all recitations of a reference value preceded by the term “about” are intended to also be a recitation of the reference value alone. Notwithstanding the preceding, in this application the term “about” has a special meaning with regard to pharmacokinetic parameters, such as area under the curve (including AUC, AUCₜ, and AUC_{∞}) Cₘₐₓ, Tₘₐₓ, and the like. When used in relationship to a value for a pharmacokinetic parameter, the term “about” means from 85% to 115% of the reference parameter.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the invention, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

Cancer cells, without wishing to be bound by theory, exhibit abnormalities, such as DNA damage, genetic instability, abnormal growth factor signaling, and abnormal or missing matrix interactions, any of which should typically induce apoptosis through the intrinsic (mitochondrial) apoptosis pathway. However, rather than respond to these apoptosis signals cancer cells survive. Often, in doing so, these cells become highly dependent on selected blocks to chronic apoptosis signals. This adaptation provides a survival mechanism for the cancer cells; however, these adaptations can also make cancer cells susceptible to particular apoptosis inducing therapies. A crucial event that commits a cell to die by intrinsic apoptosis is the permeabilization of the mitochondrial outer membrane (MOMP) and the release of molecules that activate the effector caspases. In many cases, MOMP is the point of no return in the intrinsic apoptosis pathway. Measurement of the mitochondrial response to cell treatment with the sensitizer class of BH3 containing peptides, or low doses of the activator class of BH3 peptides, allows determination of whether the cancer is “primed” to die via the intrinsic apoptotic pathway, and if so, whether the apoptosis is dependent on any particular combination of Bcl-2 anti-apoptotic proteins.

MOMP is induced only if the activator BH3 proteins, Bim and Bid, are juxtaposed in the bound state. If this is the case, then Bim and Bid are displaced from the heterodimer by the particular BH3 peptides and become free to activate Bax and Bak. When this is seen the cell is termed “primed”. By treating cells with individual selective peptides, the specific Bcl-2 family protein responsible for apoptotic blockade can be identified. A cell yielding a high apoptotic response to Noxa is said to be Mcl-1 primed, while a high response to the peptide Bad indicates that Bcl-xL or Bcl-2 provides the apoptotic block. Response to the Puma BH3 peptide reflects pan-Bcl-2 family priming. In this way, cells that are dependent on either Mcl-1 or other anti-apoptotic Bcl-2 family proteins are readily distinguished so that appropriate treatment may be tailored accordingly. The distinctions in mitochondrial response to these peptides, combinations of these peptides, or combinations of peptides and BH3 mimetic compounds, will guide the use of therapies that directly target anti-apoptotic Bcl-2 proteins or that work upstream in the intrinsic apoptosis pathway.

The Bcl-2 family proteins are the key regulators of MOMP. Their activity is linked to the onset of lymphoid and several solid tumor cancers and is believed in many cancers to be the key mediator of resistance to chemotherapy. Bcl-2 proteins are regulated by distinct protein-protein interactions between pro-survival (anti-apoptotic) and pro-apoptotic members. These interactions occur primarily through BH3 (Bcl-2 homology domain-3) mediated binding. Apoptosis-initiating signaling occurs for the most part upstream of the mitochondria and causes the translocation of short, BH3-only, Bcl-2 family members to the mitochondria where they either activate or sensitize MOMP. The activator BH3 only proteins, Bim and Bid, bind to and directly activate the effector, pro-apoptotic proteins Bax and Bak, and also bind to and inhibit the anti-apoptotic Bcl-2 family proteins, Bcl-2, Mcl-1, Bfl-1, Bcl-w and Bcl-xL. The sensitizer BH3 proteins, Bad, Bik, Noxa, Hrk, Bmf and Puma, bind only to the anti-apoptotic Bcl-2 family proteins, Bcl-2, Mcl-1, Bfl-1, Bcl-w and Bcl-xL, blocking their anti-apoptotic functions. Without wishing to be bound by theory, each sensitizer protein has a unique specificity profile. For example, Noxa (A and B) bind with high affinity to Mcl-1, Bad binds to Bcl-xL and Bcl-2 but only weakly to Mcl-1, and Puma binds well to all three targets. An anti-apoptotic function of these proteins is the sequestering of the activator BH3 protein Bim and Bid. Displacement of these activators by sensitizer peptides results in Bax/Bak-mediated apoptotic commitment. These interactions can have various outcomes, including, without limitation, homeostasis, cell death, sensitization to apoptosis, and blockade of apoptosis.

A defining feature of cancer cells in which apoptotic signaling is blocked is an accumulation of the BH3 only activator proteins at the mitochondrial surface, a result of these proteins being sequestered by the anti-apoptotic proteins. This accumulation and proximity to their effector target proteins accounts for increased sensitivity to antagonism of Bcl-2 family proteins in the “BH3 primed” state.

The value of Bcl-2 as a target in anti-tumor therapy has been well established. Briefly, without wishing to be bound by theory, as a result of aberrant phenotypes, cancer cells develop blocks in apoptosis pathways. These blocks make cancer cells both resistant to some therapies, and, surprisingly, make some cancer cells sensitive to other therapies. Bcl-2 promotes cell survival and normal cell growth, and is expressed in many types of cells including lymphocytes, neurons, and self-renewing cells, such as basal epithelial cells and hematopoietic progenitor cells in the bone marrow. Researchers have recognized that proteins in the Bcl-2 family regulate apoptosis and are key effectors of tumorigenesis (Reed, (2002) Nat Rev. Drug Discov. 1(2): 111-21). It has also been reported that Mcl-1 is a target in treating NHL, CLL, and acute mylogenous leukemia (AML) (Derenne, et al. (2002) Blood, 100: 194-99; Kitada, et al. (2004) J. Nat. Canc. Inst. 96: 642-43; Petlickovski, et al. (3018) Blood 105: 4820-28).

In many cancers, anti-apoptotic Bcl-2 proteins, block the sensitivity of tumor cells to cytostatic or apoptosis inducing drugs, and these proteins have become targets for anti-tumor therapy. BH3 mimetic compounds comprise a recently described class of small molecules that inhibits Bcl-2 family proteins are the (reviewed in Bajwa, et al. (2013) Expert Opin Ther Pat. 2012 January; 22(1): 37-55) These compounds function by inhibiting BH3 mediated protein/protein interactions among the Bcl-2 family proteins. Several studies have described BH3 mimetic small molecules that function as Bcl-2 inhibitors by blocking BH3 binding (reviewed in Billard, (2013) Mol Cancer Ther. 12(9):1691-700). Compounds with BH3 mimic function include HA-14-1 (Wang, et al. (2000) Proc. Natl. Acad. Sci. USA 97: 7124-9), Antimycin-A (Tzung, et al. (2001) Nat. Cell. Biol. 3: 183-191), BH3I-1 and BH3I-2 (Degterev, et al. (2001) Nat. Cell. Biol. 3: 173-82), and seven un-named compounds (Enyedy, et al. (2001) J. Med Chem 44: 4313-24), as well as a series of terphenyl derivatives (Kutzki, et al. (2002) J. Am. Chem. Soc. 124: 11838-9), and two new classes of molecules (Rosenberg, et al. (2004) Anal. Biochem. 328: 131-8). Compounds with selective BH3 mimic function include Bcl-2 selective activity (Ng (2014) Clin Adv Hematol Oncol. 12(4):224-9)—as well as selective Mcl-1 activity (Richard, et al. (2013) Bioorg Med Chem. 21(21):6642-9) and are in various stages of clinical development. More recently, a BH3 mimic compound has been tested in a mouse tumor model (Oltersdorf, et al. (2005) Nature 435: 677-81).

While the promise for using BH3 mimetic compounds as anti-tumor therapeutics has been recognized, to date there are no conclusive clinical reports on the efficacy of any anti-cancer drug with this mode of action. While pharmacological manipulation of the Bcl-2 family proteins is a feasible approach to achieving therapeutic benefit for cancer patients, the complexity of the network of proteins that comprise this family makes this prospect difficult. Therefore, with the large unmet medical need for treating hematological malignancies, new approaches to assessing and utilizing the detailed activity of the BH3 mimetic molecules will have value in developing this class of therapeutics.

The mitochondrial profiling assay described herein provides a predictive test for cancer treatments that work through the mitochondrial apoptosis pathway. Mitochondrial profiling uses peptides derived from pro-apoptotic BH3-only proteins and measures the degree to which MOMP occurs in a cell to determine the cell's likelihood to undergo apoptosis in response to chemotherapy (U.S. Pat. No. 8,221,966, herein incorporated by reference in its entirety). Some cancer cells, not all, are “pre-set” to undergo drug-induced apoptosis, which is induced by exposure to certain BH3 peptides. The mitochondrial depolarization following exposure to a given BH3 peptide serves as a functional biomarker of the predisposition for cellular response to pro-apoptotic cues (Pierceall et al. Mol. Cancer Ther. 12(2) 2940-9 (2013)). Analysis of whether MOMP occurs and, if so, which BH3 peptide provides the apoptotic cue allows a determination of the cell or specimen's particular chemoresistance or chemosensitivity and provides insight into the likelihood of a cancer cell to respond to treatment. This technology has demonstrated medical utility as a predictive diagnostic test for a number of cancers, including blood cancers.

Our inventive method involves the coupling of an oncology therapy and unique companion diagnostic test that is used before and during treatment to monitor treatment efficacy and predict likely continued response to treatment. This information can be used to determine the appropriateness of continuing a given treatment, and to then guide alternative treatment if required.

We have discovered a unique method for using the mitochondrial profiling technology as a pharmacodynamic marker that can determine if a cancer cell is responsive at time of initial treatment, and whether treatment is changing the cancer cell in way that shifts its responsiveness to treatment. In particular the present method provides a pharmacodynamic marker for oncology therapies that work through the mitochondrial apoptosis pathway. The pharmacodynamic marker consists of a shift in the readout between the mitochondrial profile taken before treatment and that taken at a time point during treatment and the use of that marker as a means for predicting duration of cancer patient response to treatment.

For example, cancer cells with particular dependence on particular members of the Bcl-2 family to survive can be identified by the mitochondrial profiling assay. These cancer cells are expected to be sensitive to particular therapies. For instance, cancer cells that are dependent on the Bcl-2 protein, but not the Mcl-1 protein, will be responsive to a drug that specifically targets that protein, such as the Abbott ABT-199 drug (a). The sensitivity of the cancer to a particular therapeutic can be monitored during treatment by performing the mitochondrial profile at various time points during the course of treatment. If for example, the mitochondrial profile shifts during the course of treatment to indicate sensitivity to a different BH3 peptide, e.g. a Bcl-xl dependence, then the treatment would be changed to a drug that targets Bcl-xl, e.g. Abbott ABT-263 drug (b). If for example, the profile shift indicates a dependence on the Mcl-1 protein, as indicated by response to the NOXA peptide, a drug that targets Mcl-1, e.g. Eutropics EU-5148 (E), would be appropriate. This information will guide the use of the appropriate drugs that have apoptosis independent mechanism of action in conferring cytotoxicity through perturbation of metabolic pathways such as the electron transport inhibitors, for example, rotenone, the uncoupling reagents, for example dinitrophenol, or the oxidative phosphorylation inhibitors, for example, oligomycin.

In some embodiments, a cell yielding a high apoptotic response to Noxa (A or B) is Mcl-1 primed, while a high response to the peptide Bad indicates that Bcl-xL or Bcl-2 provides the apoptotic block. In some embodiments, Puma reflects pan-Bcl-2 family priming. In this way, cells that are dependent on either Mcl-1 or Bcl-xL, on both proteins, or on several Bcl-2 family members are readily distinguished so that appropriate treatment may be tailored accordingly. The distinctions in mitochondrial response to these peptides guides the use of therapies that are known to work through pathways that funnel into either Mcl-1 or Bcl-xL affected intrinsic signaling. The use of a Bcl-2 inhibiting or a Mcl-1 inhibiting compound may be indicated in such cases. In some embodiments, the present methods also indicate or contraindicate therapies that target entities upstream of Mcl-1 or Bcl-xL.

Additionally, the test can identify cancers that during treatment shift in their sensitivity to any class of drugs that directly or indirectly induce apoptosis through the mitochondrial apoptosis pathway. This is done when the signature mitochondria profile is shown to correlate to a particular therapy.

The method proposed here is especially significant because of the severity and importance of cancer, and in particular, multiple myeloma (MM), a devastating malignancy that originates in antibody-secreting bone marrow plasma cells. The National Cancer Institute estimates that there are 63,000 cases of MM in the US, with nearly 22,000 new cases and approximately 11,000 deaths per year. The clinical course of the disease is highly variable and difficult to predict. The disease remains incurable, relapse is inevitable, and current therapies often cause considerable toxicities. Precisely targeted therapies with low toxicity would significantly enhance the repertoire available to doctors and patients for the treatment of this lethal disease.

A test that could predict MM patient response to particular drugs would improve efficacy of first and second line treatment strategies. For example, patients with poor prognoses could be steered toward experimental treatments at an earlier stage. While there are a variety of clinical indicators and cytogenetic markers used for the assessment of MM disease status and to follow disease progression, these are insufficiently precise to guide therapy. No prognostic tests exist for predicting MM patient response to any given chemotherapeutic regime, and consequently this remains a critical unmet need. In addition, patients that do respond to standard of care relapse with a high frequency. A test that could predict relapse and could guide next line of treatment would be very useful.

Previous studies have demonstrated that the mitochondrial profiling assay is predictive of response to treatment in a number of cancers including MM, acute myeloid leukemia (AML), chronic lymphocytic leukemia (CLL), Diffuse large B-cell lymphoma (DLBCL) and other cancers. In these studies mitochondrial profiling is performed before treatment is administered to patients, and the test results are correlated to observed patient responses and patient outcomes. However, in our novel method the utility of the assay is extended to provide a pharmacodynamic marker that will help anticipate relapse and provide a means to prescribe best dosing regimens and treatment options. The method measures shifts in the mitochondrial profile that occur in response to treatment by comparing the mitochondiral profile measurements taken before treatment with those taken after treatment has started. Our approach involves utilization of our diagnostic assay at the early stages of treatment to identify on-target activity, and throughout treatment to predict patient response during the course of treatment. Further, our novel method employs the application of an algorithm to the readout from the mitochondrial profiling which allows a more accurate association of the predisposition of a cell to undergo apoptosis and the cancer's sensitivity to treatment.

### Mitochondrial Profiling

A critical area of focus in cancer treatment is understanding, detecting, and controlling mitochondrial function in response to drugs and other treatments. Events occurring at the mitochondrial surface determine the ability of the cancer cell to respond to apoptosis-inducing cancer therapy. Mitochondria therefore represent a critical node for understanding how to selectively kill cancer cells while preserving non-cancer cells. Mitochondria can be evaluated to determine a cell's state using our panel of sensitizer BH3-peptides, which are selective antagonists of anti-apoptotic BCL-2 family members. Mitochondria that are predisposed to drug-induced apoptosis are dependent on anti-apoptotic protein function to prevent mitochondrial outer membrane permeabilization (MOMP), and for example, an increase in MOMP (as demonstrated by a shift from red to green emission in the JC-1 dye readout) is observed when the cells are exposed to sensitizer BH3 peptides.

The present invention uses the determination of a cancer cell's predisposition to undergo apoptosis to elucidate the cancer's susceptibility to a particular treatment. One way this can be done is by using a panel of peptides derived from BH3 domains of BH3-only proteins, or small molecule mimetics of these peptides that selectively antagonize individual BCL-2 family members BCL-2, BCL-XL, BCL-w, MCL-1 and BFL-1. Antiapoptotic family members may be distinguished from each other based on their affinity for individual BH3 domains. For instance, BCL-XL may be distinguished from BCL-2 and BCL-w by its greater affinity for HRK BH3. In contrast MCL-1 does not bind BAD BH3 (Opferman et al. 2003).

If a cell is pre-set to undergo drug-induced apoptosis (e.g. the cell is dependent on Bcl-2 polypeptide activity for survival), the assay can also be used to identify the specific Bcl-2 proteins that are responsible for apoptotic block. By directly assessing the function of the Bcl-2 proteins in the context of the mitochondria, mitochondrial profiling provides a distinctly advantageous approach relative to existing diagnostic technology, which relies solely on the correlation between genetic markers and a disease state. Mitochondrial profiling uses a panel of BH3 domain peptides, for example, those recited in Table 1. In addition to the BH3 peptides recited in Table 1, BH3 mimetics can be used in the panel. For example, a BH3 mimetic compound targeting Bcl-2 and Bcl-xL (e.g Abt-263) or a BH3 mimetic compounds targeting Mcl-1 (e.g. EU-51aa48) may be used. Each of antiapoptotic proteins BCL-2, BCL-XL, MCL-1, BFL-1 and BCL-w bear a unique pattern of interaction with this panel of proteins. As detailed below, the cellular response to the peptides is measured, for example, by the occurrence of MOMP or cytochrome C release.

TABLE 1
BH3 peptides

| | | SEQ |
|---|---|---|
| | | ID |
| BH3 peptide | Amino Acid Sequence | NO |
| BID | EDIIRNIARHLAQVGDSMDR | 1 |
| BIM | MRPEIWIAQELRRIGDEFNA | 2 |
| BID mut | EDIIRNIARHAAQVGASMDR | 3 |
| BAD | NLWAAQRYGRELRRMSDEFVDSFK | 4 |
| BIK | MEGSDALALRLACIGDEMDV | 5 |
| NOXA A | AELPPEFAAQLRKIGDKVYC | 6 |
| NOXA B | PADLKDECAQLRRIGDKVNL | 7 |
| HRK | SSAAQLTAARLKALGDELHQ | 8 |
| PUMA | EQWAREIGAQLRRMADDLNA | 9 |
| BMF | HQAEVQIARKLQLIADQFHR | 10 |
| BNI | VVEGEKEVEALKKSADWVSD | 11 |
| BMF | HQAEVQIARKLQLIADQFHR | 12 |
| huBAD | NLWAAQRYGRELRRMSDEFVDSFKK | 13 |
| BADmut | LWAAQRYGREARRMSDEFEGSFKGL | 14 |

The BH3 panel can further comprise variants of the BH3 domains or mimetics thereof. For example, a BH3 domain peptide can include a peptide which includes (in whole or in part) the sequence NH2-XXXXXXIAXXLXXXGDXXXX-COOH or NH2-XXXXXXXXXXLXXXXDXXXX-COOH. The BH3 domain can comprise at least about 5, about 6, about 7, about 8, about 9, about 10, about 15, or about 20 or more amino acids of any of SEQ ID NOs: 1-14. Preferred variants are those that have conservative amino acid substitutions made at one or more predicted non-essential amino acid residues. For example, a “conservative amino acid substitution” is one in which the amino acid residue is replaced with an amino acid residue having a similar side chain. In a further embodiment, the BH3 domain peptide is an activator or a sensitizer of apoptosis. In a preferred embodiment, the BH3 domain peptide is a sensitizer.

In various embodiments, the BH3 panel comprises one or more BH3 mimetics. BH3 mimetics or analogs thereof, that may be used in the present invention include, but are not limited to, Gossypol and its analogs (e.g. Ideker et al. Genome Res. 2008), ABT-199, ABT-737 (e.g. Petros et al. Protein Sci. 2000), ABT-263 (e.g. Letai et al. Cancer Cell 2002) and their analogues (e.g. WO2005049593, U.S. Pat. Nos. 7,767,684, 7,906,505), Obatoclax (e.g. WO2004106328, WO2005117908, U.S. Pat. No. 7,425,553), EU-5148, EU-5346, EU-4030, EU-51aa48 (Eutropics), compounds that selectively inhibit Mcl-1 (e.g. WO2008131000, WO2008130970, Richard, et al. (2013) Bioorg Med Chem. 21(21):6642-9)), HA-14-1 (e.g. Wang, et al. (2000) Proc. Natl. Acad. Sci. USA 97: 7124-9), Antimycin-A (e.g. Tzung, et al. (2001) Nat. Cell. Biol. 3: 183-191), BH3I-1 and BH3I-2 (e.g. Degterev, et al. (2001) Nat. Cell. Biol. 3: 173-82), terphenyl derivatives (e.g. Kutzki, et al. (2002) J. Am. Chem. Soc. 124: 11838-9), and compounds with selective BH3 mimic function (e.g. Ng (2014) Clin Adv Hematol Oncol. 12(4):224-9.

In various embodiments, the invention comprises mitochondrial profiling in which at least two, or three, or four, or five, or six, or seven, or eight, or nine, or ten or more BH3 peptides and/or BH3 mimetics are evaluated at once. In some embodiments, the present methods comprise a multipeptide analysis, as opposed to an evaluation of a single BH3 peptide. In some embodiments, a panel of BH3 peptides and/or BH3 mimetics is screened on a single patient specimen.

In some embodiments, the mitochondrial profiling comprises use of one or more peptides or fragments thereof, wherein the peptide is one or more of BIM, BIM2A, BAD, BID, HRK, PUMA, NOXA, BMF, BIK, and PUMA2A. In some embodiments, the mitochondrial profiling comprises use of an antibody directed against one of more of BIM, BIM2A, BAD, BID, HRK, PUMA, NOXA, BMF, BIK, and PUMA2A and naturally-occurring heterodimers formed between two Bcl-2 proteins, e.g. a first Bcl-2 protein (e.g., Bim, Bid, Bad, Puma, Noxa, Bak, Hrk, Bax, or Mule) and a second Bcl-2 protein (e.g., Mcl-1, Bcl-2, Bcl-XL, Bfl-1 or Bcl-w) as described in U.S. Pat. No. 8,168,755, the contents of which are hereby incorporated by reference in their entireties. In some embodiments the mitochondrial profiling comprises use of a stapled peptide (e.g. a peptide generated through the synthetic enhancement of a 3-D alpha-helix protein segment with hydrocarbon bonds to make proteins more rigid and able to penetrate cells), as described in, for example, Verdine, et al. “Stapled Peptides for Intracellular Drug Targets” Methods in Enzymology, Volume 503 (Chap. 1), the contents of which are hereby incorporated by reference in their entireties.

In one embodiment, the peptide is used at a concentration of about 0.1 μM to about 200 μM. In some embodiments, about 0.1 μM to about 150 μM, or about 0.1 μM to about 100 μM, or about 0.1 μM to about 50 μM, or about 0.1 μM to about 10 μM, or about 0.1 μM to about 5 μM, about 1 μM to about 150 μM, or about 1 μM to about 100 μM, about 1 μM to about 50 μM, about 1 μM to about 10 μM, about 1 μM to about 5 μM, or about 10 μM to about 100 μM of the peptide is used. In some embodiments, a concentration of about 0.1 M, or about 0.5 μM, or about 1.0 μM, or about 5 μM, or about 10 μM, or about 50 μM, or about 100 μM, or about 150 μM, or about 200 μM of the peptide is used.

In various aspects, the invention provides methods of predicting sensitivity of a cell to a therapeutic agent by contacting the cell with a BH3 domain peptide and detecting MOMP both before and after contacting said cell with a therapeutic agent. In one embodiment, the mitochondrial profiling comprises subjecting a patient cancer cell or specimen to a BH3 panel, and comparing the mitochondrial profile of the patient sample to that of a test cell or specimen (e.g. from an individual without cancer, a naïve patient, or the same patient before treatment). The method may further comprise comparing the BH3 panel read-out between the patient or test sample, and correlating any differences in the mitochondrial profile of the sample to sensitivity and/or resistance to a particular treatment. In a further embodiment, an algorithm is applied to the read-outs between the patient and test samples and the results of the algorithm are correlated with any differences in sample sensitivity and/or resistance to a particular treatment. Alternatively, sensitivity of a cell to a therapeutic agent is determined by providing a mitochondrial profile of the cancer cell after contact with the therapeutic agent and comparing the mitochondrial profile to the initial profile. A shift of the mitochondrial profile in the cancer cell after treatment compared to the initial mitochondrial profile provides a pharmacodynamic marker to indicate the cancer cell's resistance or sensitivity and predict response to treatment.

Apoptosis is detected by various means known in the art, and for example, by detecting loss of mitochondrial outer membrane permeabilization (MOMP), or measuring cytochrome C release. The loss of mitochondrial outer membrane permeabilization can be measured for example, using the potentiometric dye JC-1 or dihydrorhodamine. In one embodiment, the therapeutic agent is a chemotherapeutic agent.

In one embodiment, the predisposition of a cell to undergo apoptosis is determined by measuring the amount of cytochrome C release from the mitochondria, which is a marker of apoptosis. This can be measured using standard techniques known in the art (See for example, Current Protocols in Molecular Biology, Greene Publ. Assoc. Inc. & John Wiley & Sons, Inc., Boston, Mass., 1993).

In one embodiment, the predisposition of a cell to undergo apoptosis is determined by measuring the amount of the cell's mitochondrial outer membrane permeabilization (MOMP). This can be performed using standard techniques known in the art, including those described in Bogenberger et al. (Leukemia et al. (2014) which is herein incorporated by reference in its entirety). In a non-limiting example, cells are permeabilized and incubated with a mitochondrial dye (e.g. JC-1 or dihydrorhodamine 123) and BH3 peptides with dimethyl sulfoxide or carbonyl cyanide m-chlorophenyl hydrazone (CCCP) and the degree of staining is measured.

The mitochondrial profiling comprises associating the propensity of a pro-apoptotic peptide to induce mitochondrial depolarization (% priming) and patient classification (e.g. responder/non-responder). In other embodiments, the application of an algorithm to the percent priming by any particular BH3 peptide, mimetic, or combination thereof is associated with patient classification (e.g. responder/non-responder).

Mitochondrial profiling and reagents useful for such a method is described in U.S. Pat. Nos. 7,868,133; 8,221,966; and 8,168,755 and US Patent Publication No. 2011/0130309, the contents of which are hereby incorporated by reference in their entireties.

In one aspect, the invention provides a mitochondrial profile containing a pattern of mitochondrial sensitivity to BH3 peptides taken from one or more subjects who have cancer.

In some embodiments, the invention predicts the efficacy of a cancer treatment which can include one or more of anti-cancer drugs, chemotherapy, surgery, adjuvant therapy (e.g. prior to surgery), and neoadjuvant therapy (e.g. after surgery). In another embodiment, the cancer treatment comprises one or more of a BH3 mimetic, epigenetic modifying agent, topoisomerase inhibitor, cyclin-dependent kinase inhibitor, and kinesin-spindle protein stabilizing agent. In still another embodiment, the cancer treatment comprises a proteasome inhibitor; and/or a modulator of cell cycle regulation (by way of non-limiting example, a cyclin dependent kinase inhibitor); and/or a modulator of cellular epigenetic mechanistic (by way of non-limiting example, one or more of a histone deacetylase (HDAC) (e.g. one or more of vorinostat or entinostat), azacytidine, decitabine); and/or an anthracycline or anthracenedione (by way of non-limiting example, one or more of epirubicin, doxorubicin, mitoxantrone, daunorubicin, idarubicin); and/or a platinum-based therapeutic (by way of non-limiting example, one or more of carboplatin, cisplatin, and oxaliplatin); cytarabine or a cytarabine-based chemotherapy; a BH3 mimetic (by way of non-limiting example, one or more of BCL2, BCLXL, or MCL1); an apoptotic protein; a glucocorticoid, a steroid, a monoclonal antibody, an antibody-drug conjugate, or thalidomide derivative, and an inhibitor of MCL1.

In some embodiments, the mitochondrial profiling comprises permeabilizing the patient's cancer cells, and determining or quantifying a change in mitochondrial membrane potential upon contacting the permeabilized cells with one or more BH3 domain peptides and/or one or more therapeutics. In some embodiments, the mitochondrial profiling is performed both before and during cancer treatment. These measurements, along with the clinical factors described herein, help differentiate patient response and/or patients for a variety of therapies.

In certain embodiments, the mitochondrial priming is defined by the following equation:

$$\% Priming = [ 100 * ( \frac{DMSO AUC - {Peptide}_{1} AUC}{DMSO AUC - {CCCP}_{avg} AUC} ) ] {Peptide}_{1} + [ 100 * ( \frac{DMSO AUC - {Peptid}_{2} AUC}{DMSO AUC - {CCCP}_{avg} AUC} ) ] {Peptide}_{2} + … \text{/} ( n peptides )$$

in which the AUC comprises either area under the curve or signal intensity; the DMSO comprises the baseline negative control; and the CCCP (Carbonyl cyanide m-chlorophenyl hydrazone) comprises an effector of protein synthesis by serving as uncoupling agent of the proton gradient established during the normal activity of electron carriers in the electron transport chain in the mitochondria comprises the baseline positive control. In some embodiments, the area under the curve is established by homogenous time-resolved fluorescence (HTRF). In some embodiments, the time occurs over a window from between about 0 to about 300 min to about 0 to about 30 min. In some embodiments, the area under the curve is established by fluorescence activated cell sorting (FACS). In some embodiments, the signal intensity is a single time point measurement that occurs between about 5 min and about 300 min.

In some embodiments, the method comprises analysis of a patient's clinical factor. In various embodiments, the clinical factor is one or more of age, cytogenetic status, performance, histological subclass, gender, and disease stage. In another embodiment, the method further comprises a measurement of an additional biomarker selected from mutational status, single nucleotide polymorphisms, steady state protein levels, and dynamic protein levels, which can add further specificity and/or sensitivity to the test. In another embodiment, the method further comprises predicting a clinical response in the patient. In another embodiment, the clinical response is at least about 1, about 2, about 3, or about 5 year progression/event-free survival.

In another embodiment, the method comprises conducting the mitochondrial profiling assay and one or more of a cell surface marker CD33, a cell surface marker CD34, a FLT3 mutation status, a p53 mutation status, a phosphorylation state of MEK-1 kinase, and phosphorylation of serine at position 70 of Bcl-2; and correlating to efficacy in treating cancer patients with chemotherapy. In one embodiment, the cancer patient is an AML patient. In another embodiment, the cancer patient is a MM patient.

In another embodiment, the mitochondrial profile is performed during the course of treatment. In a further embodiment, the mitochondrial profile is performed on the patient's cell or sample before and at various time points during treatment. In another embodiment, the mitochondrial profile is performed on the patient's cell or sample at various time points during treatment. In one embodiment, patient samples are taken before treatment commences (time “0”) and subsequently at any appropriate time point during or after treatment. In one embodiment, the decision to perform a subsequent mitochondrial profile in a patient is made when the patient stops responding to a current course of treatment. In another embodiment, the decision to perform a subsequent mitochondrial profile is made independently of the patient's response to treatment.

In one aspect, the mitochondrial profile is performed in vitro. In a further embodiment, the BH3 is performed in vivo. In vivo mitochondrial profiling may be performed in any appropriate method, and for example, by engrafting the cells to a model organism, such as mouse. In one embodiment, the mouse is a SCID mouse. In one embodiment, engrafted cells express a luminescent marker, thereby allowing optical tracking of the cells in vivo (see for example, Runnels et al. J. Biomed. Opt. 16(1) January 11(2011)).

In one aspect, the invention provides applying an algorithm to the results of the mitochondrial profiling, and analyzing the pattern and/or degree of response in the mitochondrial profile to predict the cell or specimen sensitivity to treatment. In one embodiment, sequential biomarker algorithms derived from assessment of the mitochondrial profile are applied to classify a patient according to likely response to treatment. In one embodiment, the algorithm is applied to predict the shift in cell response (e.g. sensitivity or resistance) as measured in the mitochondrial profile. In one non-limiting example, BIM and NOXA metrics are critical determinants of 5-Aza response. (See Bogenberger et al. Leukemia (2014) the contents of which are herein incorporated by reference in its entirety).

### Exemplary Clinical Decisions

In some embodiments, the methods described herein are useful in the evaluation of a patient, for example, for evaluating diagnosis, prognosis, and response to treatment. In various aspects, the present invention comprises evaluating a tumor or hematological cancer. In various embodiments, the evaluation may be selected from diagnosis, prognosis, and response to treatment.

Diagnosis refers to the process of attempting to determine or identify a possible disease or disorder, such as, for example, cancer. Prognosis refers to predicting a likely outcome of a disease or disorder, such as, for example, cancer. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment. Responses to treatment can be, by way of non-limiting example, pathological complete response, survival, and progression free survival, time to progression, probability of recurrence.

As used herein, the term “neoadjuvant therapy” refers to treatment given as a first step to shrink a tumor before the main treatment, which is usually surgery, is given. Examples of neoadjuvant therapy include chemotherapy, radiation therapy, and hormone therapy. In some embodiments, the present methods direct a patient's treatment to include neoadjuvant therapy. For example, a patient that is scored to be responsive to a specific treatment may receive such treatment as neoadjuvant therapy. In some embodiments, neoadjuvant therapy means chemotherapy administered to cancer patients prior to surgery. In some embodiments, neoadjuvant therapy means an agent, including those described herein, administered to cancer patients prior to surgery. Further, the present methods may direct the identity of a neoadjuvant therapy, by way of non-limiting example, as a treatment that induces and/or operates in a pro-apoptotic manner or one that does not. In one embodiment, the present methods may indicate that a patient will not be or will be less responsive to a specific treatment and therefore such a patient may not receive such treatment as neoadjuvant therapy. Accordingly, in some embodiments, the present methods provide for providing or withholding neoadjuvant therapy according to a patient's likely response. In this way, a patient's quality of life, and the cost of case, may be improved.

As used herein, the term “adjuvant therapy” refers to additional cancer treatment given after the primary treatment to lower the risk that the cancer will come back. Adjuvant therapy may include chemotherapy, radiation therapy, hormone therapy, targeted therapy, or biological therapy. In some embodiments, the present methods direct a patient's treatment to include adjuvant therapy. For example, a patient that is scored to be responsive to a specific treatment may receive such treatment as adjuvant therapy. Further, the present methods may direct the identity of an adjuvant therapy, by way of non-limiting example, as a treatment that induces and/or operates in a pro-apoptotic manner or one that does not. In one embodiment, the present methods may indicate that a patient will not be or will be less responsive to a specific treatment and therefore such a patient may not receive such treatment as adjuvant therapy. Accordingly, in some embodiments, the present methods provide for providing or withholding adjuvant therapy according to a patient's likely response. In this way, a patient's quality of life, and the cost of care, may be improved.

In various embodiments, the present methods direct a clinical decision regarding whether a patient is to receive a specific treatment. In one embodiment, the present methods are predictive of a positive response to neoadjuvant and/or adjuvant chemotherapy or a non-responsiveness to neoadjuvant and/or adjuvant chemotherapy. In one embodiment, the present methods are predictive of a positive response to a pro-apoptotic agent or an agent that operates via apoptosis and/or an agent that does not operate via apoptosis or a non-responsiveness to apoptotic effector agent and/or an agent that does not operate via apoptosis. In various embodiments, the present invention directs the treatment of a cancer patient, including, for example, what type of treatment should be administered or withheld.

In one embodiment, a comparison of the data generated in the mitochondrial profile performed at various time points during treatment shows a change in profile readout indicating a change in the cancer's sensitivity to a particular treatment. In one embodiment, the determination of a cancer's change in sensitivity to a particular treatment is used to re-classify the patient and to guide the course of future treatment.

In one embodiment, the determination of the sensitivity or resistance of a patient's cancer cell to a particular therapeutic is used to classify the patient into a treatment or prognosis group. In some non-limiting examples, patients are classified into groups designated as cure, relapse, no complete response, complete response, refractory to initial therapy, responder, non-responder, high likelihood of response, or low likelihood of response. In further embodiments, analysis of the mitochondrial profiling and patient classification direct a clinical decision regarding treatment, such as, for example, switching from one therapeutic to another, a change in dose of therapeutic, or administration of a different type of treatment (e.g. surgery, radiation, allogenic bone marrow or stem cell transplant). In a further embodiment, clinical decision is directed by the analysis of a change in cancer sensitivity, classification, and consideration of clinical factors, such as age and/or cytogenetic status. In various embodiments, a cancer treatment is administered or withheld based on the methods described herein. Exemplary treatments include surgical resection, radiation therapy (including the use of the compounds as described herein as, or in combination with, radiosensitizing agents), chemotherapy, pharmacodynamic therapy, targeted therapy, immunotherapy, and supportive therapy (e.g., painkillers, diuretics, antidiuretics, antivirals, antibiotics, nutritional supplements, anemia therapeutics, blood clotting therapeutics, bone therapeutics, and psychiatric and psychological therapeutics).

In one embodiment, the present methods direct a clinical decision regarding whether a patient is to receive adjuvant therapy after primary, main or initial treatment, including, without limitation, a single sole adjuvant therapy. By way of non-limiting example, adjuvant therapy may be an additional treatment usually given after surgery where all detectable disease has been removed, but where there remains a statistical risk of relapse due to occult disease.

In an exemplary embodiment, the present method will indicate a likelihood of response to a specific treatment. For example, in some embodiments, the present methods indicate a high or low likelihood of response to a pro-apoptotic agent and/or an agent that operates via apoptosis and/or an agent that operates via apoptosis driven by direct protein modulation. In various embodiments, exemplary pro-apoptotic agents and/or agents that operate via apoptosis and/or an agent that operates via apoptosis driven by direct protein modulation include ABT-263 (Navitoclax), and obatoclax, WEP, bortezomib, and carfilzomib. In some embodiments, the present methods indicate a high or low likelihood of response to an agent that does not operate via apoptosis and/or an agent that does not operate via apoptosis driven by direct protein modulation. In various embodiments, exemplary agents that do not operate via apoptosis include kinesin spindle protein inhibitors, cyclin-dependent kinase inhibitor, Arsenic Trioxide (TRISENOX), MEK inhibitors, pomolidomide, azacytidine, decitibine, vorinostat, entinostat, dinaciclib, gemtuzumab, BTK inhibitors, PI3 kinase delta inhibitors, lenolidimide, anthracyclines, cytarabine, melphalam, Aky inhibitors, mTOR inhibitors.

In an exemplary embodiment, the present method will indicate whether a patient is to receive a pro-apoptotic agent or an agent that operates via apoptosis for cancer treatment. In another exemplary embodiment, the present method will indicate whether a patient is to receive an agent that does not operate via apoptosis.

In a specific embodiment, the present methods are useful in predicting a cancer patient's response to any of the treatments (including agents) described herein. In an exemplary embodiment, the present invention predicts a cancer patient's likelihood of response to chemotherapy and comprises an evaluation of the mitochondiral profile, age profile and cytogenetic factors of the patient.

### Exemplary Treatments

In exemplary embodiments, the invention selects a treatment agent. Examples of such agents include, but are not limited to, one or more of anti-cancer drugs, chemotherapy, surgery, adjuvant therapy, and neoadjuvant therapy. In one embodiment, the cancer treatment is one or more of a BH3 mimetic, epigenetic modifying agent, topoisomerase inhibitor, cyclin-dependent kinase inhibitor, and kinesin-spindle protein stabilizing agent. In another embodiment, the cancer treatment is a proteasome inhibitor; and/or a modulator of cell cycle regulation (by way of non-limiting example, a cyclin dependent kinase inhibitor); and/or a modulator of cellular epigenetic mechanistic (by way of non-limiting example, one or more of a histone deacetylase (HDAC) (e.g. one or more of vorinostat or entinostat), azacytidine, decitabine); and/or an anthracycline or anthracenedione (by way of non-limiting example, one or more of epirubicin, doxorubicin, mitoxantrone, daunorubicin, idarubicin); and/or a platinum-based therapeutic (by way of non-limiting example, one or more of carboplatin, cisplatin, and oxaliplatin); cytarabine or a cytarabine-based chemotherapy; a BH3 mimetic (by way of non-limiting example, one or more of BCL2, BCLXL, MCL1, Abt-263, EU-51aa48, EU-5346, and EU-5148); a glucocorticoid, a steroid, a monoclonal antibody, an antibody-drug conjugate, a thalidomide derivative, and an inhibitor of MCL1.

In various embodiments, the invention pertains to cancer treatments including, without limitation, those described in US Patent Publication No. US 2012-0225851 and International Patent Publication No. WO 2012/122370, the contents of which are hereby incorporated by reference in their entireties.

In various embodiments, the invention pertains to cancer treatments including, without limitation, one or more of alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation, dacogen, velcade, and pharmaceutically acceptable salts, acids or derivatives of any of the above.

### Exemplary Detection Methods

In one embodiment, the predisposition of a cell to undergo apoptosis is determined by measuring mitochondrial outer membrane permeability or detecting cytochrome C release, both hallmarks of apoptosis. In one embodiment, the predisposition of a cell to undergo apoptosis is determined by measuring the amount of cytochrome C release from the mitochondria, which is a marker of apoptosis. This can be measured using standard techniques known in the art (See for example, Current Protocols in Molecular Biology, Greene Publ. Assoc. Inc. & John Wiley & Sons, Inc., Boston, Mass., 1993).

In various embodiments, the present methods comprise evaluating the cytogenetic status of a cell (e.g. evaluating a presence, absence, or level of a protein and/or a nucleic acid). In various embodiments, the present methods comprise evaluating a presence, absence, or level of a protein and/or a nucleic acid which can enhance the specificity and/or sensitivity of mitochondrial profiling. In some embodiments, the evaluating is of a marker for patient response. In some embodiments, the present methods comprise measurement using one or more of immunohistochemical staining, western blotting, in cell western, immunofluorescent staining, ELISA, and fluorescent activating cell sorting (FACS), bioluminescence, fluorescent marker detection, or any other method described herein or known in the art. The present methods may comprise contacting an antibody with a tumor specimen (e.g. biopsy or tissue or body fluid) to identify an epitope that is specific to the tissue or body fluid and that is indicative of a state of a cancer.

There are generally two strategies used for detection of epitopes on antigens in body fluids or tissues, direct methods and indirect methods. The direct method comprises a one-step staining, and may involve a labeled antibody (e.g. FITC conjugated antiserum) reacting directly with the antigen in a body fluid or tissue sample. The indirect method comprises an unlabeled primary antibody that reacts with the body fluid or tissue antigen, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, NY, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, Calif.).

In various embodiments, antibodies include whole antibodies and/or any antigen binding fragment (e.g., an antigen-binding portion) and/or single chains of these (e.g. an antibody comprising at least two heavy (H) chains and two light (L) chains inter-connected by disulfide bonds, an Fab fragment, a monovalent fragment consisting of the V_{L}, V_{H}, C_{L} and CH1 domains; a F(ab)₂ fragment, a bivalent fragment including two Fab fragments linked by a disulfide bridge at the hinge region; a Fd fragment consisting of the V_{H} and CH1 domains; a Fv fragment consisting of the V_{L} and V_{H} domains of a single arm of an antibody; and the like). In various embodiments, polyclonal and monoclonal antibodies are useful, as are isolated human or humanized antibodies, or functional fragments thereof.

Standard assays to evaluate the binding ability of the antibodies toward the target of various species are known in the art, including for example, ELISAs, western blots and RIAs. The binding kinetics (e.g., binding affinity) of antibodies also can be assessed by standard assays known in the art, such as by Biacore analysis.

In another embodiment, the measurement comprises evaluating a presence, absence, or level of a nucleic acid. A person skilled in the art will appreciate that a number of methods can be used to detect or quantify the DNA/RNA levels of appropriate markers.

Gene expression can be measured using, for example, low-to-mid-plex techniques, including but not limited to reporter gene assays, Northern blot, fluorescent in situ hybridization (FISH), and reverse transcription PCR (RT-PCR). Gene expression can also be measured using, for example, higher-plex techniques, including but not limited, serial analysis of gene expression (SAGE), DNA microarrays. Tiling array, RNA-Seq/whole transcriptome shotgun sequencing (WTSS), high-throughput sequencing, multiplex PCR, multiplex ligation-dependent probe amplification (MLPA), DNA sequencing by ligation, and Luminex/XMAP. A person skilled in the art will appreciate that a number of methods can be used to detect or quantify the level of RNA products of the biomarkers within a sample, including arrays, such as microarrays, RT-PCR (including quantitative PCR), nuclease protection assays and Northern blot analyses.

### Exemplary Cancers and Patients

In some embodiments the invention provides a method for determining a cancer treatment and/or comprises a patient's tumor or cancer cell specimen. A cancer or tumor refers to an uncontrolled growth of cells and/or abnormal increased cell survival and/or inhibition of apoptosis which interferes with the normal functioning of the bodily organs and systems. A subject that has a cancer or a tumor is a subject having objectively measurable cancer cells present in the subject's body. Included in this invention are benign and malignant cancers, as well as dormant tumors or micrometastatses. Cancers which migrate from their original location and seed vital organs can eventually lead to the death of the subject through the functional deterioration of the affected organs.

In various embodiments, the invention is applicable to pre-metastatic cancer, or metastatic cancer. Metastasis refers to the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are often detected through the sole or combined use of magnetic resonance imaging (MRI) scans, computed tomography (CT) scans, blood and platelet counts, liver function studies, chest X-rays and bone scans in addition to the monitoring of specific symptoms.

The methods described herein are directed toward the prognosis of cancer, diagnosis of cancer, treatment of cancer, and/or the diagnosis, prognosis, treatment, prevention or amelioration of growth, progression, and/or metastases of malignancies and proliferative disorders associated with increased cell survival, or the inhibition of apoptosis. In some embodiments, the cancer is a hematologic cancer, including, but not limited to, acute myelogenous leukemia (AML), multiple myeloma, follicular lymphoma, acute lymphoblastic leukemia (ALL), chronic lymphocytic leukemia, and non-Hodgkin's lymphoma including, but not limited to, mantle cell lymphoma and diffuse large B-cell lymphoma. In some embodiments, the cancer is a solid tumor, including, but not limited to, non-small lung cell carcinoma, ovarian cancer, and melanoma.

In some embodiments, the invention relates to one or more of the following cancers: acute lymphoblastic leukemia (ALL), acute myeloid leukemia (AML), adrenocortical carcinoma, AIDS-related cancers, anal cancer, appendix cancer, astrocytoma (e.g. childhood cerebellar or cerebral), basal-cell carcinoma, bile duct cancer, bladder cancer, bone tumor (e.g. osteosarcoma, malignant fibrous histiocytoma), brainstem glioma, brain cancer, brain tumors (e.g. cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt's lymphoma, carcinoid tumors, central nervous system lymphomas, cerebellar astrocytoma, cervical cancer, chronic lymphocytic leukemia (CLL), chronic myelogenous leukemia (CML), chronic myeloproliferative disorders, colon cancer, cutaneous t-cell lymphoma, desmoplastic small round cell tumor, endometrial cancer, ependymoma, esophageal cancer, Ewing's sarcoma, extracranial germ cell tumor, extragonadal germ cell tumor, extrahepatic bile duct cancer, eye cancer, gallbladder cancer, gastric (stomach) cancer, gastrointestinal stromal tumor (GIST), germ cell tumor (e.g. extracranial, extragonadal, ovarian), gestational trophoblastic tumor, gliomas (e.g. brain stem, cerebral astrocytoma, visual pathway and hypothalamic), gastric carcinoid, head and neck cancer, heart cancer, hepatocellular (liver) cancer, hypopharyngeal cancer, hypothalamic and visual pathway glioma, intraocular melanoma, islet cell carcinoma (endocrine pancreas), kidney cancer (renal cell cancer), laryngeal cancer, leukemias (e.g. acute lymphocytic leukemia, acute myelogenous leukemia, chronic lymphocytic leukemia, chronic myeloid leukemia, hairy cell), lip and oral cavity cancer, liposarcoma, liver cancer, lung cancer (e.g. non-small cell, small cell), lymphoma (e.g. AIDS-related, Burkitt, cutaneous T-cell Hodgkin, non-Hodgkin, primary central nervous system), medulloblastoma, melanoma, Merkel cell carcinoma, mesothelioma, metastatic squamous neck cancer, mouth cancer, multiple endocrine neoplasia syndrome, multiple myeloma, mycosis fungoides, myelodysplastic syndromes, myelodysplastic/myeloproliferative diseases, myelogenous leukemia, myeloid leukemia, myeloid leukemia, myeloproliferative disorders, chronic, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma, ovarian cancer, pancreatic cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal tumors, pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, retinoblastoma, rhabdomyosarcoma, salivary gland cancer, sarcoma (e.g. Ewing family, Kaposi, soft tissue, uterine), Sezary syndrome, skin cancer (e.g. nonmelanoma, melanoma, merkel cell), small cell lung cancer, small intestine cancer, soft tissue sarcoma, squamous cell carcinoma, squamous neck cancer, stomach cancer, supratentorial primitive neuroectodermal tumor, t-cell lymphoma, testicular cancer, throat cancer, thymoma and thymic carcinoma, thyroid cancer, trophoblastic tumors, ureter and renal pelvis cancers, urethral cancer, uterine cancer, uterine sarcoma, vaginal cancer, visual pathway and hypothalamic glioma, vulvar cancer, Waldenström macroglobulinemia, and Wilms tumor.

In one embodiment, the cancer is multiple myeloma (MM). In one embodiment, the cancer is AML. AML is the second most common leukemia, with approximately 13,000 newly diagnosed cases and 9,000 deaths annually in the US. Although approved therapies exist, the prognosis of many leukemia patients is poor and the likelihood of successful treatment is low. The current standard of care for AML is induction cytosine arabinoside (ara-C) in combination with an anthracycline agent (such as, for example, daunarubicin, idarubicine or mitoxantrone). This therapeutic regimen is typically followed by administration of high dose cytarabine and/or stem cell transplantation. These treatments have improved outcome in young patients. Progress has also been made in the treatment of acute promyelocytic leukemia, where targeted therapy with all-trans retinoic acid (ATRA) or arsenic trioxide have resulted in excellent survival rates. However, patients over 60, a population which represents the vast majority of AML cases, remain a therapeutic enigma. Although 65-85% of patients initially respond to existing treatments, 65% of such responders undergo relapse, and many patients succumb to the disease. For at least this reason and because the afore-mentioned treatments may have severe side effects, the inventive predictive test can guide use of the treatment that mitigates these litigations. In some embodiments, the present invention improves the likelihood of successful treatment by matching the right patient to the right treatment. Further, there are currently no tests to predict AML patient response to treatment.

The term subject, as used herein unless otherwise defined, is a mammal, e.g., a human, mouse, rat, hamster, guinea pig, dog, cat, horse, cow, goat, sheep, pig, or non-human primate, such as a monkey, chimpanzee, or baboon. The terms “subject” and “patient” are used interchangeably.

### Exemplary Specimens

In some embodiments, the present invention includes the measurement of a tumor specimen, including biopsy or surgical specimen samples. In some embodiments, the specimen is selected from a frozen tumor tissue specimen, cultured cells, circulating tumor cells, and a formalin-fixed paraffin-embedded tumor tissue specimen (e.g. for antibody based mitochondrial profiling). In some embodiments, the biopsy is a human biopsy. In various embodiments, the biopsy is any one of a frozen tumor tissue specimen, cultured cells, circulating tumor cells, and a formalin-fixed paraffin-embedded tumor tissue specimen (e.g. for antibody based mitochondrial profiling).

In some embodiments, the tumor specimen may be a biopsy sample, such as a frozen tumor tissue (cryosection) specimen. As is known in the art, a cryosection may employ a cryostat, which comprises a microtome inside a freezer. The surgical specimen is placed on a metal tissue disc which is then secured in a chuck and frozen rapidly to about −20° C. to about −30° C. The specimen is embedded in a gel like medium consisting of, for example, poly ethylene glycol and polyvinyl alcohol. The frozen tissue is cut frozen with the microtome portion of the cryostat, and the section is optionally picked up on a glass slide and stained.

In some embodiments, the tumor specimen may be a biopsy sample, such as cultured cells. These cells may be processed using the usual cell culture techniques that are known in the art. These cells may be circulating tumor cells.

In some embodiments, the tumor specimen may be a biopsy sample, such as a formalin-fixed paraffin-embedded (FFPE) tumor tissue specimen. As is known in the art, a biopsy specimen may be placed in a container with formalin (a mixture of water and formaldehyde) or some other fluid to preserve it. The tissue sample may be placed into a mold with hot paraffin wax. The wax cools to form a solid block that protects the tissue. This paraffin wax block with the embedded tissue is placed on a microtome, which cuts very thin slices of the tissue.

In certain embodiments, the tumor specimen (or biopsy) contains less than 100 mg of tissue, or in certain embodiments, contains about 50 mg of tissue or less. The tumor specimen (or biopsy) may contain from about 20 mg to about 50 mg of tissue, such as about 35 mg of tissue.

The tissue may be obtained, for example, as one or more (e.g., 1, 2, 3, 4, or 5) needle biopsies (e.g., using a 14-gauge needle or other suitable size). In some embodiments, the biopsy is a fine-needle aspiration in which a long, thin needle is inserted into a suspicious area and a syringe is used to draw out fluid and cells for analysis. In some embodiments, the biopsy is a core needle biopsy in which a large needle with a cutting tip is used during core needle biopsy to draw a column of tissue out of a suspicious area. In some embodiments, the biopsy is a vacuum-assisted biopsy in which a suction device increases the amount of fluid and cells that is extracted through the needle. In some embodiments, the biopsy is an image-guided biopsy in which a needle biopsy is combined with an imaging procedure, such as, for example, X ray, computerized tomography (CT), magnetic resonance imaging (MRI) or ultrasound. In other embodiments, the sample may be obtained via a device such as the MAMMOTOME® biopsy system, which is a laser guided, vacuum-assisted biopsy system for breast biopsy.

In certain embodiments, the specimen is a human tumor-derived cell line. In certain embodiments, the specimen is a cancer stem cell. In other embodiments, the specimen is derived from the biopsy of a solid tumor, such as, for example, a biopsy of a colorectal, breast, prostate, lung, pancreatic, renal, or ovarian primary tumor.

In certain embodiments, the specimen is of epithelial origin. In some embodiments, the epithelial specimen is enriched by selection from a biopsy sample with an anti-epithelial cell adhesion molecule (EpCAM) or other epithelial cell binding antibody bound to solid matrix or bead.

In certain embodiments, the specimen is of mesenchymal origin. In some embodiments, the mesenchymal specimen is enriched by selection from a biopsy sample with a neural cell adhesion molecule (N-CAM) or neuropilin or other mesenchymal cell binding antibody bound to a solid matrix or bead.

In certain embodiments, the specimen is derived from the biopsy of a non-solid tumor, such as, for example, any of the cancer described herein. In specific embodiments, the specimen is derived from the biopsy of a patient with multiple myeloma, acute myelogenous leukemia, acute lymphocytic leukemia, chronic lymphogenous leukemia, mantle cell lymphoma, diffuse large B-cell lymphoma, and non-Hodgkin's lymphoma. In a specific embodiment, the specimen is a multiple myeloma cell that is enriched by selection from a biopsy sample with an anti-CD138 antibody bound to a solid matrix or bead. In a specific embodiment, the specimen is an acute myelogenous leukemia cell that is enriched by binding to a CD45-directed antibody. In a specific embodiment, the specimen is a chronic lymphogenous leukemia or diffuse large B-cell lymphoma that is enriched by non-B cell depletion.

In some embodiments, the specimen is derived from a circulating tumor cell.

### Exemplary Clinical Factors and Additional Biomarkers

In some embodiments, the invention comprises the evaluation of clinical factors. In some embodiments, the invention comprises an evaluation of mitochondrial profiling and/or clinical factors to assess a patient response. In some embodiments, a clinical factor that provides patient response information in combination with a mitochondrial profiling study may not be linked to apoptosis. In some embodiments, a clinical factor is non-apoptosis affecting.

In one embodiment, the clinical factor is one or more of age, cytogenetic status, performance, histological subclass, gender, and disease stage

In one embodiment, the clinical factor is age. In one embodiment, the patient age profile is classified as over about 10, or over about 20, or over about 30, or over about 40, or over about 50, or over about 60, or over about 70, or over about 80 years old.

In one embodiment, the clinical factor is cytogenetic status. In some cancers, such as Wilms tumor and retinoblastoma, for example, gene deletions or inactivations are responsible for initiating cancer progression, as chromosomal regions associated with tumor suppressors are commonly deleted or mutated. For example, deletions, inversions, and translocations are commonly detected in chromosome region 9p21 in gliomas, non-small-cell lung cancers, leukemias, and melanomas. Without wishing to be bound by theory, these chromosomal changes may inactivate the tumor suppressor cyclin-dependent kinase inhibitor 2A. Along with these deletions of specific genes, large portions of chromosomes can also be lost. For instance, chromosomes 1p and 16q are commonly lost in solid tumor cells. Gene duplications and increases in gene copy numbers can also contribute to cancer and can be detected with transcriptional analysis or copy number variation arrays. For example, the chromosomal region 12q13-q14 is amplified in many sarcomas. This chromosomal region encodes a binding protein called MDM2, which is known to bind to a tumor suppressor called p53. When MDM2 is amplified, it prevents p53 from regulating cell growth, which can result in tumor formation. Further, certain breast cancers are associated with overexpression and increases in copy number of the ERBB2 gene, which codes for human epidermal growth factor receptor 2. Also, gains in chromosomal number, such as chromosomes 1q and 3q, are also associated with increased cancer risk.

Cytogenetic status can be measured in a variety of manners known in the art. For example, FISH, traditional karyotyping, and virtual karyotyping (e.g. comparative genomic hybridization arrays, CGH and single nucleotide polymorphism arrays) may be used. For example, FISH may be used to assess chromosome rearrangement at specific loci and these phenomenon are associated with disease risk status. In some embodiments, the cytogentic status is favorable, intermediate, or unfavorable.

In one embodiment, the clinical factor is performance. Performance status can be quantified using any system and methods for scoring a patient's performance status are known in the art. The measure is often used to determine whether a patient can receive chemotherapy, adjustment of dose adjustment, and to determine intensity of palliative care. There are various scoring systems, including the Karnofsky score and the Zubrod score. Parallel scoring systems include the Global Assessment of Functioning (GAF) score, which has been incorporated as the fifth axis of the Diagnostic and Statistical Manual (DSM) of psychiatry. Higher performance status (e.g., at least 80%, or at least 70% using the Karnofsky scoring system) may indicate treatment to prevent progression of the disease state, and enhance the patient's ability to accept chemotherapy and/or radiation treatment. For example, in these embodiments, the patient is ambulatory and capable of self care. In other embodiments, the evaluation is indicative of a patient with a low performance status (e.g., less than 50%, less than 30%, or less than 20% using the Karnofsky scoring system), so as to allow conventional radiotherapy and/or chemotherapy to be tolerated. In these embodiments, the patient is largely confined to bed or chair and is disabled even for self-care.

The Karnofsky score runs from 100 to 0, where 100 is “perfect” health and 0 is death. The score may be employed at intervals of 10, where: 100% is normal, no complaints, no signs of disease; 90% is capable of normal activity, few symptoms or signs of disease, 80% is normal activity with some difficulty, some symptoms or signs; 70% is caring for self, not capable of normal activity or work; 60% is requiring some help, can take care of most personal requirements; 50% requires help often, requires frequent medical care; 40% is disabled, requires special care and help; 30% is severely disabled, hospital admission indicated but no risk of death; 20% is very ill, urgently requiring admission, requires supportive measures or treatment; and 10% is moribund, rapidly progressive fatal disease processes.

The Zubrod scoring system for performance status includes: 0, fully active, able to carry on all pre-disease performance without restriction; 1, restricted in physically strenuous activity but ambulatory and able to carry out work of a light or sedentary nature, e.g., light house work, office work; 2, ambulatory and capable of all self-care but unable to carry out any work activities, up and about more than 50% of waking hours; 3, capable of only limited self-care, confined to bed or chair more than 50% of waking hours; 4, completely disabled, cannot carry on any self-care, totally confined to bed or chair; 5, dead.

In one embodiment, the clinical factor is histological subclass. In some embodiments, histological samples of tumors are graded according to Elston & Ellis, Histopathology, 1991, 19:403-10, the contents of which are hereby incorporated by reference in their entirety.

In one embodiment, the clinical factor is gender. In one embodiment, the gender is male. In another embodiment the gender is female.

In one embodiment, the clinical factor is disease stage. By way of non-limiting example, using the overall stage grouping, Stage I cancers are localized to one part of the body; Stage II cancers are locally advanced, as are Stage III cancers. Whether a cancer is designated as Stage II or Stage III can depend on the specific type of cancer. In one non-limiting example, Hodgkin's disease, Stage II indicates affected lymph nodes on only one side of the diaphragm, whereas Stage III indicates affected lymph nodes above and below the diaphragm. The specific criteria for Stages II and III therefore differ according to diagnosis. Stage IV cancers have often metastasized, or spread to other organs or throughout the body.

In some embodiments, the clinical factor is the French-American-British (FAB) classification system for hematologic diseases (e.g. indicating the presence of dysmyelopoiesis and the quantification of myeloblasts and erythroblasts). In one embodiment, the FAB for acute lymphoblastic leukemias is L1-L3, or for acute myeloid leukemias is MO-M7.

In another embodiment, the method further comprises a measurement of an additional biomarker selected from mutational status, single nucleotide polymorphisms, steady state protein levels, and dynamic protein levels. In another embodiment, the method further comprises predicting a clinical response in the patient. In another embodiment, the clinical response is about 1, about 2, about 3, or about 5 year progression/event-free survival.

A variety of clinical factors have been identified, such as age profile and performance status. A number of static measurements of diagnosis have also been utilized, such as cytogenetics and molecular events including, without limitation, mutations in the genes MLL, AML/ETO, Flt3-ITD, NPM1 (NPMc+), CEBPα, IDH1, IDH2, RUNX1, ras, and WT1 and in the epigenetic modifying genes TET2 and ASXL, as well as changes in the cell signaling protein profile.

Further, in some embodiments, the any one of the following clinical factors may be useful in the methods described herein: gender; genetic risk factors; family history; personal history; race and ethnicity; features of the certain tissues; various benign conditions (e.g. non-proliferative lesions); previous chest radiation; carcinogen exposure and the like.

Further still, in some embodiments, the any one of the following clinical factors may be useful in the methods described herein: one or more of a cell surface marker CD33, a cell surface marker CD34, a FLT3 mutation status, a p53 mutation status, a phosphorylation state of MEK-1 kinase, and phosphorylation of serine at position 70 of Bcl-2.

In some embodiments, the clinical factor is expression levels of the cytokines, including, without limitation, interleukin-6. In some embodiments, interleukin-6 levels will correlate with likelihood of response in MM patients, including a poor patient prognosis or a good patient prognosis.

In another embodiment, the method comprises measuring the mitochondrial profiling assay of a cell expressing one or more of a cell surface marker CD33, a cell surface marker CD34, a FLT3 mutation status, a p53 mutation status, a phosphorylation state of MEK-1 kinase, and phosphorylation of serine at position 70 of Bcl-2; and correlating to efficacy in treating cancer patients with chemotherapy.

In still another embodiment, the cancer is AML and/or MM and the clinical factor is age profile and/or cytogenetic status; or the cancer is AML and/or MM and the cancer treatment is cytarabine or cytarabine-based chemotherapy and/or azacytidine, or the cancer treatment is cytarabine or cytarabine-based chemotherapy and/or azacytidine and the clinical factor is age profile and/or cytogenetic status, or the cancer treatment is cytarabine or cytarabine-based chemotherapy and/or azacytidine; the cancer is AML and/or MM; and the clinical factor is age profile and/or cytogenetic status.

The invention also provides kits that can simplify the evaluation of tumor or cancer cell specimens. A typical kit of the invention comprises various reagents including, for example, one or more agents to detect a BH3 peptide. A kit may also comprise one or more of reagents for detection, including those useful in various detection methods, such as, for example, antibodies. The kit can further comprise materials necessary for the evaluation, including welled plates, syringes, and the like. The kit can further comprise a label or printed instructions instructing the use of described reagents. The kit can further comprise a treatment to be tested.

This invention is further illustrated by the following non-limiting examples.

### EXAMPLES

### Example 1: Mitochondrial Profiling Assay

The mitochondrial profiling assay relies on the use of the sensitizer or activator BH3 domain peptides to probe cancer cell mitochondria. A mitochondrial response signature to any one or any class of BH3 peptide indicates a dependence on a particular anti-apoptotic Bcl-2 family protein. Peptides derived from the sensitizer proteins can induce apoptotic signaling in vitro, and each sensitizer protein has a unique specificity profile (Table 2). For example, two peptides (Noxa, Mule) interact only with Mcl-1, and thus cause permeabilization only in Mcl-1 dependent mitochondria. Bcl-2 (and Bcl-xL) dependent mitochondria display unique sensitivity to the BAD peptide. Other peptides such as Puma show broad spectrum affinity and their activity provides a general index of cell “priming” or Bcl-2 family dependence. These peptides, though poor in vivo drugs due to extremely poor pharmacologic properties, are excellent as in vitro probes for characterizing the Bcl-2 dependence of a cell and as positive controls for the behavior of ideal Mcl-1 inhibitors.

Table 2 shows the BH3 domain binding pattern of various BH3 containing peptides and Mimetics. Binding affinities (K_{d} in nM) between BH3 peptides (columns) and their cognate proteins (rows) are shown.

TABLE 2

| | BIM | BAD | NOXA | PUMA | Mimetic 1 | Mimetic 2 |
|---|---|---|---|---|---|---|
| | BH3 | BH3 | BH3 | BH3 | (ABT-263) | (EU-5346) |
| BCL-2 | <10 nM | 11 nM | NA | 18 nM | 0.02 nM | >10 uM |
| BCL-XL | <10 nM | <10 nM | NA | <10 nM | 0.05 nM | >10 uM |
| MCL-1 | <10 nM | NA | 19 nM | <10 nM | 520 nM | 450 nM |

Measurement of the mitochondrial response to exposure to the sensitizer class of BH3 containing peptides allows determination of whether the cancer is “primed” to die via the intrinsic apoptotic pathway, and if so, whether the apoptosis is dependent on the Bcl-2/Bcl-xL or Mcl-1 pathways.

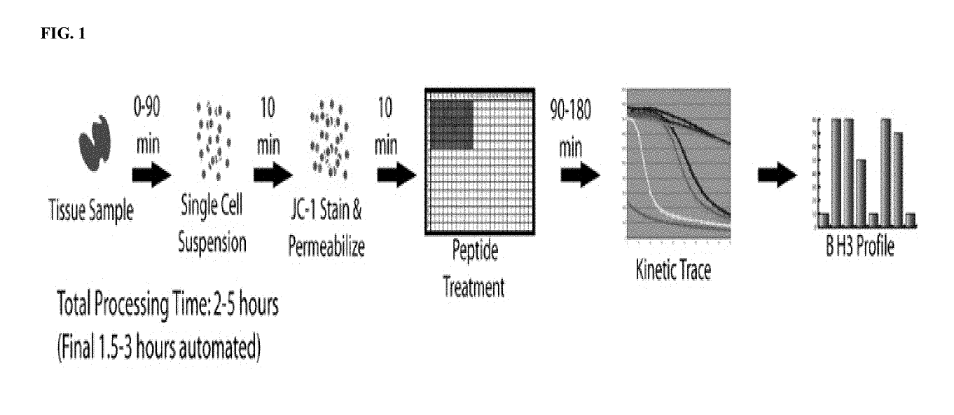

The plate-based assay format is highly sensitive, requiring small numbers of cells (FIG. 1). A FACS-based format may be used for biopsied samples that cannot easily be purified from their starting tissue preparations.

In another application, the method can be used to engraft MM cells representing each of the three categories into SCID mice and then treat with the same battery of compounds as in cell culture. Correlation of the response observed in the engrafted mice to the mitochondrial profile will demonstrate the predictive value of the mitochondrial profiling assay in vivo. Our early studies have shown that the mitochondrial profile readout does predict efficacy of the Bcl-2 restricted or Mcl-1 active compounds in vitro, and we will look for changes in the mitochondrial profile of the MM cells during the course of treatment. Detecting changes in the mitochondrial profile will forecast drug resistance to some treatments and sensitivity to others, and portend utility of the assay for future clinical use.

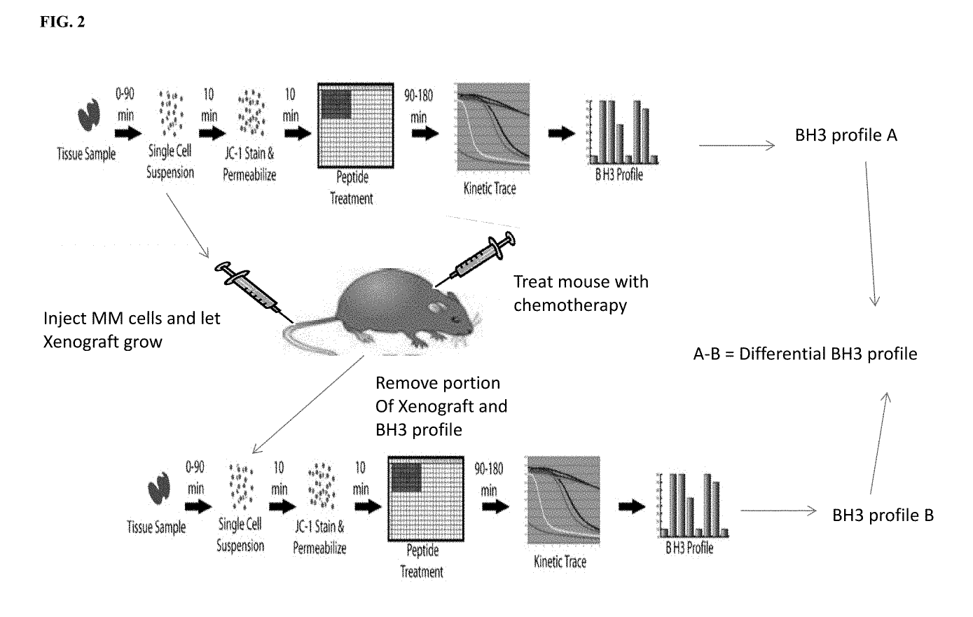

The work flow for differential mitochondrial profiling is provided in FIG. 2. Briefly, cells from patients are mitochondrial profiled as described above and then engrafted into mice. During and following treatment with chemotherapy the engrafted cancer cells are removed at various intervals from the mouse by mandibular bleeds and then mitochondrial profiled. The difference between the profiles at different treatment times is used to assess on target activity and the likelihood of further response to treatment.

BH3 Assay:

The mitochondrial profiling assay was carried out in three steps: (1) cell preparation and counting, (2) cell permeabilization and peptide treatment, and (3) fluorescent readout (FIG. 1). Cells are suspended in Mitochondria loading Buffer with 0.005% digitonin, loaded with the cationic dye JC-1 (1 μM), and treated with 100 μM of one of the BH3 domain peptides: Bim, Bad, Noxa, and Puma. MOMP is followed by full mitochondrial membrane depolarization (ΔΨm), which is measured by treating the cells with the ionophore FCCP (p-trifluoromethoxy carbonyl cyanide phenyl hydrazone). Peptide (and FCCP) addition results in a decrease in membrane potential in suitably primed cells and is measured as a decrease in JC-1 fluorescence in a 384 well plate on a TecanGenios plate reader using an excitation of 535 nM and an emission of 590 nm. Cells were treated in culture with the compound (e.g. EU-4030, EU-5148, or ABT-263) at concentrations ranging from 0.01 μM to 50 μM for 48 hours.

Tables 3 and 4 show the percent priming of various cell lines, as determined by measuring the signal intensity of the JC-1 dye which is an indicator of mitochondrial depolarization. Cell lines were grown in culture (2×10⁵ per sample) in 96 well plates and treated with 0.05 μM to 50 μM of EU-5148, ABT-263, or Obatoclax for 48 hours. Viability was measured using an MTS assay. The IC50 is in μM.

TABLE 3

| | Mcl-1 Priming | Bcl-2 Priming | | | |
|---|---|---|---|---|---|
| | State (NOXA | State (BAD | | | |
| Cell Line | Signal) | Signal) | EU-5148 | ABT-263 | Obatoclax |
| NCI-H929 | 74 (HIGH) | 57 (HIGH) | 2.9 | 4 | 1 |
| Bcl-2 1863 | 22 (LOW) | 64 (HIGH) | 3.4 | 0.9 | 2.8 |
| Mcl-1 1780 | 75 (HIGH) | 23 (LOW) | 2.7 | 17 | 1.5 |
| DHL10 | Bax/Bak (—) | Bax/Bak (—) | >25 | >25 | 0.8 |

TABLE 4

| | Mcl-1 Priming | Bcl-2 Priming | | | |
|---|---|---|---|---|---|
| | State (NOXA | State (BAD | | | |
| Cell Line | Signal) | Signal) | EU-5148 | ABT-263 | Obatoclax |
| DHL6 | 18 (LOW) | 77 (HIGH) | 7.8 | <<1 | 1.2 |
| NCI-H929 | 74 (HIGH) | 57 (HIGH) | 2.9 | 4 | 1 |
| LPN3 | 58 (HIGH) | 3 (LOW) | 2.6 | 4.1 | N/A |
| DHL10 | Bax/Bak (—) | Bax/Bak (—) | >25 | >25 | 0.8 |
| Bcl-2 1863 | 1 (LOW) | 34 (HIGH) | 2.9 | 4 | 1 |
| Mcl-1 1780 | 71 (HIGH) | 9 (LOW) | 6.5 | 17 | 1.5 |

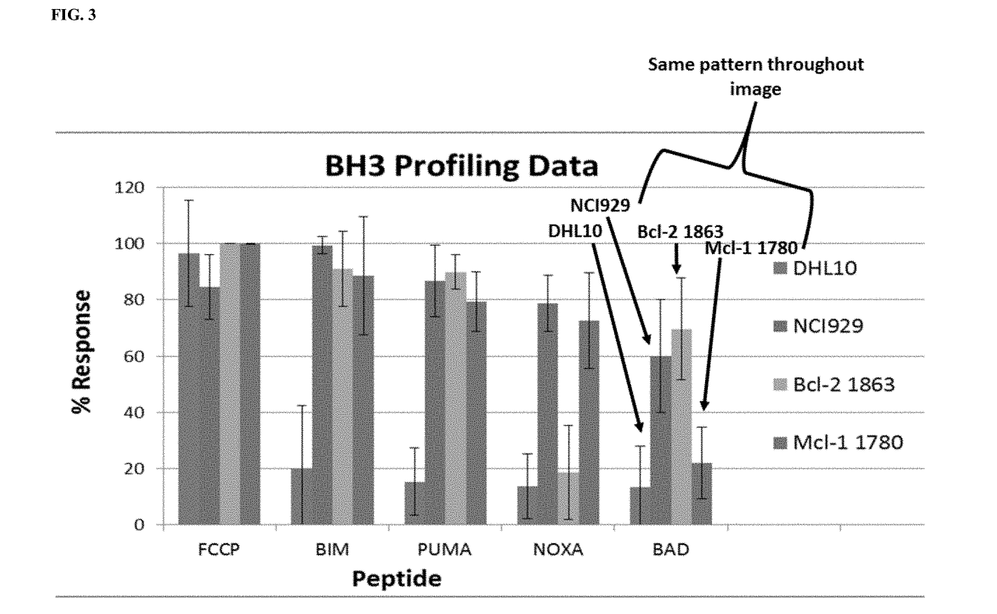

FIG. 3 shows the mitochondrial response (MOMP) to exposure to BH3 peptides. The mitochondrial profiles of cells that are Mcl-1 primed (NCI-H), Bcl-2 primed (DHL-6), or unprimed (DHL-10) are indicated as a percentage of the positive signal, Bim peptide, or FCCP in Bax, Bak deficient cells. This unprimed pattern is also seen in cells with functional Bax/Bak.

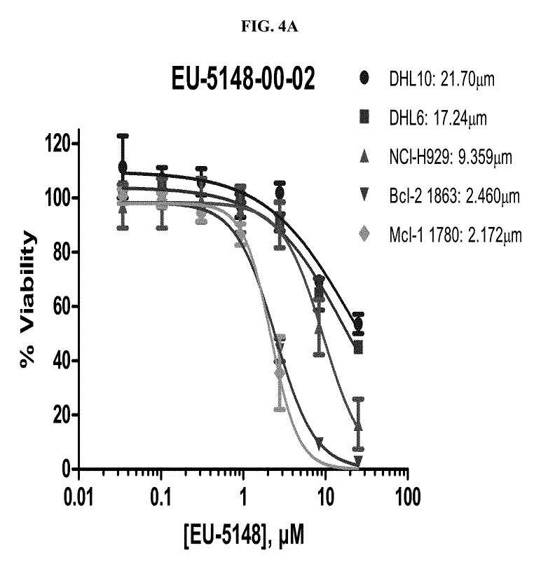

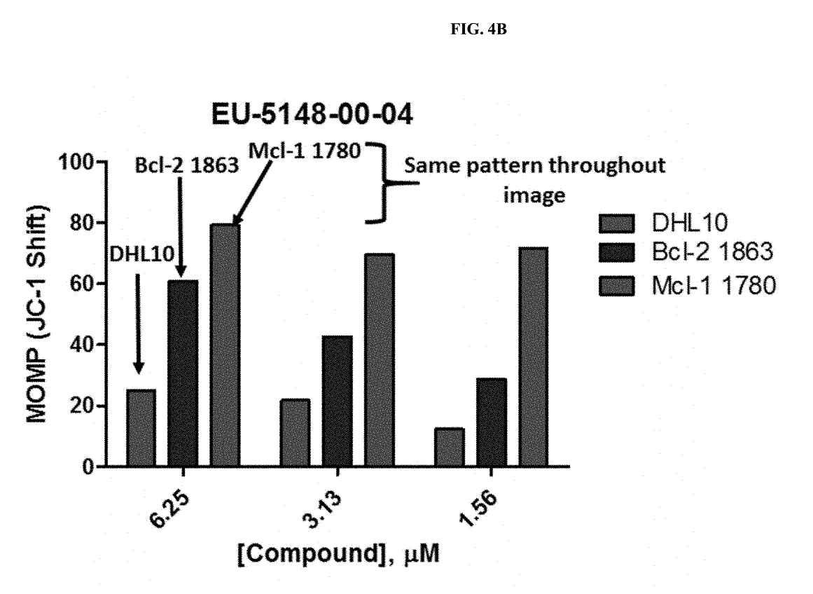

FIG. 4A shows the extent of cell killing observed correlates with the degree of Mcl-1 priming of that cell line as determined by mitochondrial profiling. EU-5148 has comparable activity (48 hours) to MLN9708 in many of the NSLC cancer cell lines treated.

FIG. 4B shows the extent of MOMP in response to Mcl-1 BH3 mimetic EU5149 observed may be correlated with the degree of Mcl-1 priming of that cell line as determined by mitochondrial profiling. Cells were prepared for the Praedicare Dx assay and the EU-5148 compound was used as the analyte. The readout is the shift in JC1 signal after 90 minutes.

### Example 2—Correlation of In Vitro Potency of Bcl-2 and Mcl-2 Inhibitors and Standard Chemotherapy to Mitochondrial Profiling Classification

Myeloma cell lines will be tested by the mitochondrial profiling assay as previously described. Cell lines fall into the following categories determined by mitochondrial profiling: (a) predominantly Mcl-1 primed (b) predominantly Bcl-2/Bcl-xL primed or (c) poorly primed. Cells lines representative of each of these classifications have been engineered to express the GFP and Luciferase genes using the Lentivirus infection as previously described. These cell lines will be tested for response to ABT-263, EU-4030, and EU-5148 as single agents or in combination with Bortezomib. Responsive and non-responsive cell lines will be monitored by the mitochondrial profiling assay before and after (in the case of non-responsive cell lines) treatment.

Cell Death Response to Bcl-2 or Mcl-1 Targeted Therapy:

Cancer cells collected from patients are determined to have a particular mitochondrial profile, and are tested for response to Bcl-2 targeted therapeutic compound. For instance, the compound EU-5148, which targets Mcl-1, has selective cell-killing activity in cells that are Mcl-1 primed (Mcl-1 1780), as determined by mitochondrial response to Noxa BH3 peptide in the mitochondrial profiling assay. The ABT-263 compound, on the other hand, is ineffective in these cells but effective in Bcl-2 primed cells (Bcl-2 1863). If the cancer cells can be primed by more than one anti-apoptotic, the pattern still instructs the use of the appropriate therapeutic target.

Cells determined to be non-primed can be so for a number of reasons, but do not respond well to therapies that target Bcl-2 proteins directly, or those that induce intrinsic apoptosis by other mechanisms. For example, the DHL-10 cells that are deficient in the Bax-Bak proteins are not responsive to EU-5148, or ABT-263. This is expected given their mechanism of action. Obatoclax, however, is effective at killing DHL-10 cells, demonstrating its off-target activity; this has also been noted elsewhere. Three MM cell lines expressing Luciferase have been categorized as Mcl-1(LPN3 and OPM-2), Bcl-2(SKMM.1), or poorly primed (OPM1). These will be tested for response to drugs in vitro and used for xenografts.

### Example 3—Detection of Tumor Progression in Mice by Bioluminescence Imaging

Mice were injected with 75 mg/kg D-luciferin, anesthetized, and imaged 10 minutes after substrate injection. Total body luminescence was determined using a standardized region of interest encompassing the entire mouse using the Living Images software package (Caliper Life Sciences).

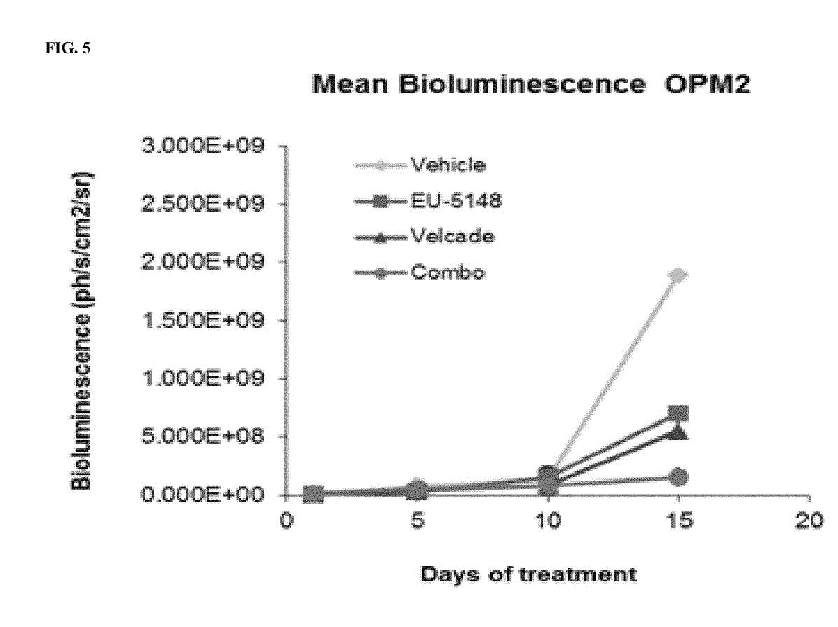

As shown in FIG. 5 a mean tumor burden reduction was observed after treatment with EU-5148, velcade, or a combination of the two compared with vehicle-only treatment. OPM2/Luciferase cells were transferred to SCID mice and allowed to reach tumor burden. Xenografted mice were treated with EU-5148 (20 mpk IV, 3×/week), velcade for (1 mpk IV, 3×/week), or a combination of these treatments. We observed a mean tumor burden reduction of 63%, as measured by bioluminescence imaging, after 15 days EU-5148 treatment. The combination treatment of EU-5148 with Velcade results in 92% reduction in tumor cell burden over same time period.

### Example 4: Correlation of Mitochondrial Profile and MM Tumor Cell Response in In Vivo Murine Model

To determine whether there is a correlation between the mitochondrial profile and Multiple Myeloma (MM) tumor cell response to targeted and non-targeted treatments in an in vivo mouse model, preliminary animal studies are performed. Bioluminescence imaging (BLI), which measures the luciferase catalyzed signal in mice and enables the longitudinal studies of the changes in tumor volume and response to treatment in individual animals over time, is used to monitor the changes in tumor volume during the course of treatment.

BH3 Assay:

Cell lines are grown under standard conditions. Multiple myeloma cell lines to study include: MM1S, OPM1, OPM2, NCI-H929, INA-6, RPMI-8226, U266B1, U266B2, and several others. The mitochondrial profiling assay is carried out in three steps: (1) cell preparation and counting, (2) cell permeabilization and peptide treatment, and (3) fluorescent readout (FIG. 1). Cells are suspended in Mitochondria loading Buffer with 0.005% digitonin and loaded with the cationic dye JC-1 (1 μM), and treated with 100 μM of one of the BH3 domain peptides: Bim, Bad, Noxa, and Puma. MOMP is followed by full mitochondrial membrane depolarization (ΔΨm), which is measured by treating the cells with the ionophore FCCP (p-trifluoromethoxy carbonyl cyanide phenyl hydrazone). Peptide (and FCCP) addition results in a decrease in membrane potential in suitably primed cells and is measured as a decrease in JC-1 fluorescence in a 384 well plate on a TecanGenios plate reader using an excitation of 535 nM and an emission of 590 nm.

Xenografted mice are treated with BH3 mimetic compounds and monitored. Mitochondrial profiled Luc-GFP-puro engineered MM cell lines representative of the following categories: (a) Mcl-1 primed, (b) Bcl-2 primed, or (c) not primed, will be used to engraft Cg-Prkdc^{scid}Il2rg^{tm1WJ1}/SzJ (NSG) immunodeficient mice. These animals have severe adaptive and innate immune deficiency with a complete absence of the IL-2 gamma chain and have been used successfully for engraftment of a diversity of solid tumor and hematologic malignancies.

Luc-GFP-puro-MM cells (Mcl-1 primed, Bcl-2 primed, or unprimed) will be injected into the tail vein of 40 seven to nine week old female NSG mice and tumor burden will be quantified by bioluminescence imaging. Mice with established disease will be defined by logarithmically increasing bioluminescence. These mice with established disease (assuming ˜80% take rate) will be randomly divided into groups: EU5148, Velcade, Combination, and vehicle alone.

### Example 5: Correlation of MM Tumor Response to Mitochondrial Profiling During Time Course

Multiple Myeloma Profiles:

To date, we have obtained Mitochondrial profiles from 14 multiple myeloma or plasma cell leukemia patients. Each of the samples tested came from patients treated with one of several different Velcade/Bortezomib combination regimens. The samples listed in Table 5 are viably frozen from a tissue bank or from fresh bone marrow delivered within a few hours of the biopsy. CD138 cells are purified from either source using two similar procedures that have been optimized for each respective tissue type. Profiles on frozen cells are performed immediately following purification. Profiles on fresh cells are performed immediately or on viably frozen CD138 positive cells.

TABLE 5

| Patient No. | Δ M-Spike | % Priming |
|---|---|---|
| 3188T | 93.03 | 99.6 |
| 3201T | 87.50 | 87.0 |
| 3187T | 65.22 | 76.1 |
| 3039T | 64.71 | 76.8 |
| 3098T | 62.50 | 70.6 |
| 3191T | 57.69 | 50.3 |
| 3221T | 52.38 | 68.0 |
| 3161T | 12.90 | 34.0 |
| 3213T | −10.00 | 40.0 |

Correlation to Bortezomib Response:

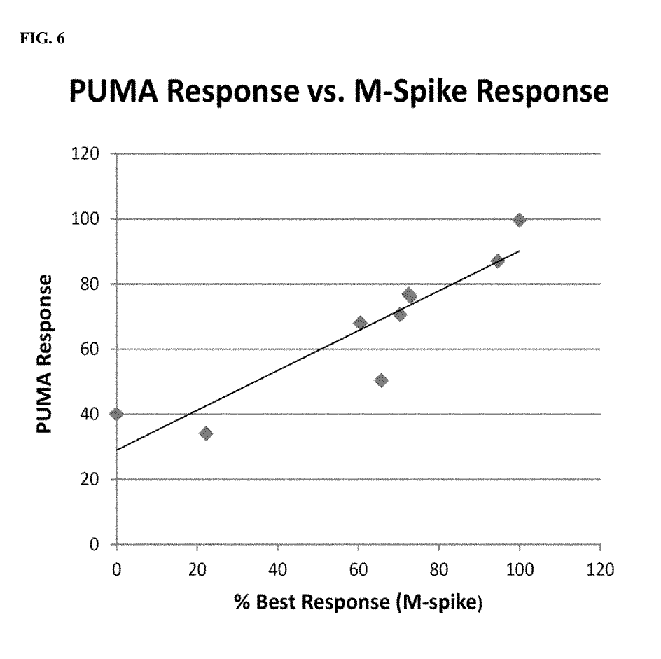

The primary indicator of Bortezomib responsiveness for multiple myeloma is the general primed state of the cell. This is indicated by the mitochondrial response to the PUMA peptide, which antagonizes all anti-apoptotic Bcl-2 family members. FIG. 6 shows the patient response to Velcade combination treatment as predicted by mitochondrial profiling. CD138+ cells were collected from bone marrow before treatment. The response to PUMA peptide was measured as an indication of a “primed state”. The difference in measurement of pre- and post-treatment M protein is used as the patient response criterion. The PUMA response values are represented as a percentage of the difference between the DMSO mitochondrial response and the FCCP mitochondrial response.

Patient response to Bortezomib treatment is measured as a change in the change in M-protein over the course of treatment. M-protein is an indication of myeloma activity and is widely used diagnostic marker of the severity of the disease. The patient M-protein response is converted to a percentage of the best response seen among the group of patients tested. The M-spike response is calculated as a percent decrease in M-protein over a given treatment period. In Table 5, the M-spike Max column shows M-protein levels at the beginning of treatment. The M-spike Min column shows the M-protein levels at the end of treatment. The percent decrease is calculated with the following equation.

$$\% Decrease = \frac{{Mspike}_{Max} - {Mspike}_{Min}}{{Mspike}_{Max}} \times 100$$

The percent best response is used to normalize the data to a fixed scale where 100% is the best response and 0% is the worst response. It is calculated with the equation below.

$$\% Best Response = \frac{\% decrease - \% {decrease}_{worst}}{\% {decrease}_{best} - \% {decrease}_{worst}} \times 100$$

As shown in FIG. 6, there is a correlation between the percent best response in M-spike and PUMA response in MM patients.

### Example 6: Detecting the Shift in the Mitochondrial Profile Over Time Course of Treatment

Cancer cells collected from a patient undergoing Velcade based treatment were mitochondrial profiled at three time points during the course of treatment (October 2010, January 2011, and May 2011). The profile was used to monitor the apoptotic predisposition of the CD-138 positive MM cells during treatment. As shown in Table 6, the signal generated by the PUMA peptide remained consistent during the time course of treatment indicating the cells remained in a “primed state” and would be advised to continue to receive treatment. The reduction in M-spike over the time course indicates that this course of action would be the correct treatment. A loss of the priming, as indicated by the reduced PUMA signal here would direct the physician to withdraw from Velcade and switch to cytotoxic drugs that are less reliant on the Bcl-2 proteins for effectiveness such as Doxil Thalidomide, or bendamustine treatments.

TABLE 6

| | | M-Spike | PUMA |
|---|---|---|---|
| TB# | Date | g/dl | % + control |
| 3098T | Oct. 13, 2010 | 1.7 | 67% |
| 3098T2 | Jan. 27, 2011 | 0.8 | 70% |
| 3098T3 | May 5, 2011 | 0.3 | 65% |

In a similar manner, patient treatment can be guided by shifts in the Mitochondrial profiling readouts. For instance a shift to a stronger Noxa signal, indicating increased Mcl-1 dependence for survival, is correlated to a shift towards sensitivity to vorinostat mylotarg combination treatment. The occurrence of this shift in the readout during standard of care (7+3) treatment of AML would direct a change in treatment to the vorinostat mylotarg regimin. Likewise, the use of BH3 mimetic class of drugs with specificity for Mcl-1 would be prescribed when a shift to the Noxa peptide readout. Such a shift during treatment with Bcl-2 targeted mimetics, such as the Bcl-2 selective ABT-199 (see attached) would call for treatment options that target Mcl-1, including antibodies targeting 11-6 or Mcl-1 targeted BH3 mimetics.

Mitochondrial profile readout algorithms that provide the best correlates to changing sensitivities are determined both in pre-clinical studies in xenografted mice and in clinical studies. In addition to the BH3 peptides described a series of BH3 mimetics that comprise a wider range of activities against individual and combinations of anti-apoptotic proteins are used for this purpose.

### Example 7: Combination Treatment

Possible novel MM treatments include combinations of drugs to treat each of the three categories of MM cell lines. Recent study indicates the likely importance of combining BH3 mimetics, including those against Mcl-1, with Velcade®. Velcade® has been shown to upregulate Mcl-1 by reducing the normal proteosomal degradation of the protein. Velcade® in combination with Revlamid (lenalidomide) and Dexamethasone is becoming the standard of care for the treatment of MM patients. Treatments to be studied will include using Velcade® in combination with the Mcl-1 selective compound EU-5148.

We will also assess the ability of the Mcl-1 inhibitor to enhance, or potentially rescue the activity of ABT-263, in animals engrafted with Bcl-2 dependent MM cells that may shift to the Mcl-1 dependent profile during treatment.

### Example 8: Correlation of Activity of Multimarkers with Cell Priming

Further insights to patient response to therapy will be generated by associating the MOMP response that results from exposure to compounds that antagonize certain combinations of pro-apoptotic and anti-apoptotic proteins. For example, MOMP in response to Mcl-1 antagonizing compound EU5148 will predict patient response to that compound, or to other Mcl-1 perturbing treatments (e.g. ant-Il-6 antibodies). In addition the readout from this compound will predict the response to other treatments that do not directly perturb the Mcl-1 proteins. For example, a combination treatment of Vorinostat and Mylotarg® (gemtuzumab ozogamicin) may be administered for AML that is predicted to be Mcl-dependent.

Compounds with dual specificity, for instance those that antagonize both Mcl-1 and Bfl-w (also called AP-1), will also be correlated to patient response. The response to compounds that have different established anti-apoptotic binding ranges will also be used to provide unique combinations of anti-apoptotic protein perturbations, increasing the range of combinations of perturbations afforded by the BH3 peptide perturbations previously described in the art.

Further, analysis of the degrees of activity of given BH3 peptides or mimetics, or the different combinations of peptide or mimetic activity in mitochondrial profiling may be more predictive of therapeutic response than the correlation of a single peptide or mimetic with efficacy. The overall balance of the activity of pro- and antiapoptotic BH3 peptides may be used to predict a patient's response to treatment.

With an increased range of perturbations observable, and the application of an appropriate algorithm, the likelihood of spotting unique mitochondrial profiles that correlate with patient response to individual or combination treatments will be enhanced. The ability to monitor unique/subtle changes in the readouts during a course of treatment will enable establishing pharmacodynamic biomarkers for guiding treatment adjustments

### Example 9: Differential Induction of MOMP by BH3 Mimetics

To test whether BH3 mimetics can induce MOMP, two compounds, A and B were used as competing ligands in the mitochondrial profiling assay and induction measured via flow cytometry. Cells were pertubated by washing and resuspending them in Newmeyer buffer. The novel compound treatments were prepared by diluting the peptide stocks and compound in Newmeyer buffer, and drug titrations were first prepared in DMSO before dilution in buffer. DMSO and CCCP were assayed as negative and positive controls, respectively, along with Bim (0.1 uM), Compound A (1.0 uM), Compound A (0.1 uM), Compound A (0.0 uM), Compound B (1.0 uM), Compound B (0.1 uM), Compound B (0.0 uM), NOXA (100 uM), Puma (10 uM), HRK (100 uM), BAD (100 uM), and BID (uM). Digitonin and oligomycin were added to all tubes followed by peptide and compound dilutions. Cells were then added and incubated for 2:15 hours at room temperature, in order for cell permeabilization and mitochondrial depolarization to occur. JC-1 dye was prepared in Newmeyer buffer and added to cells; one tube of cells was stained with propidium iodide (PI) as a permeabilization control. After 45 minutes of incubation with JC-1, cells were analyzed on a BD FACSCanto II. Cells were gated based on 4 nested gating parameters: 1) permeabilization (as determined by PI staining), 2) side- and forward-scatter (to ensure only singlet cells were analyzed) 3) AML blast population was identified as CD45 intermediate CD3 and CD20 negative 4) JC-1 red staining. The mean JC-1 red fluorescence was then used to calculate % depolarization as compared to DMSO (negative) and CCCP (positive) controls.

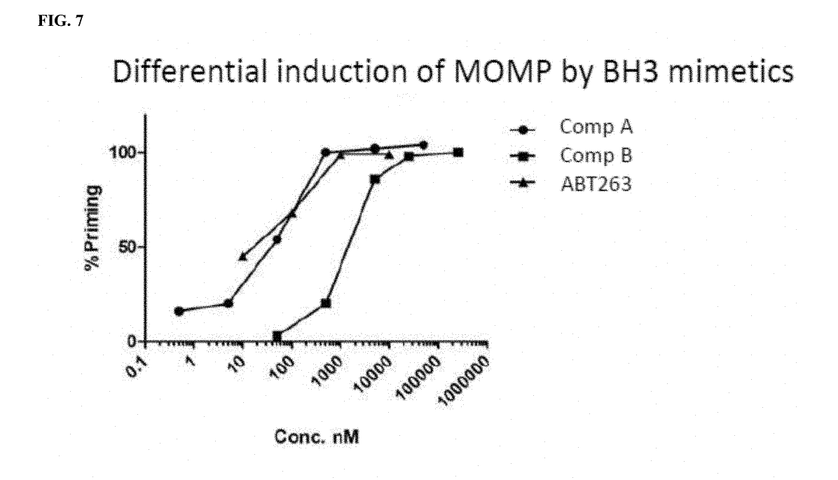

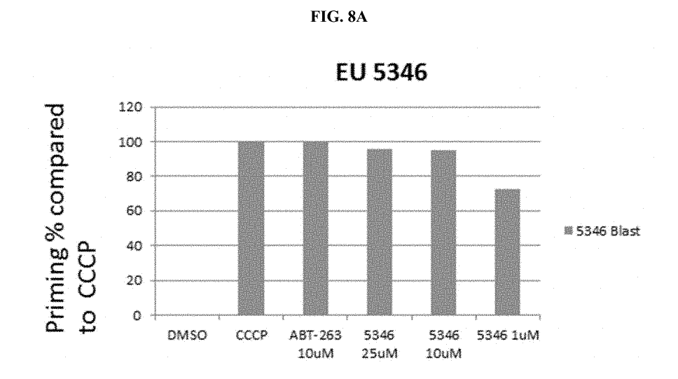

FIG. 7 shows flow cytometry-based assay for detecting MOMP caused by novel compounds. As shown in the figure, both compounds induced MOMP in the blast cell population of AML patient sample, with Compound A showing induction similar to that of ABT263.

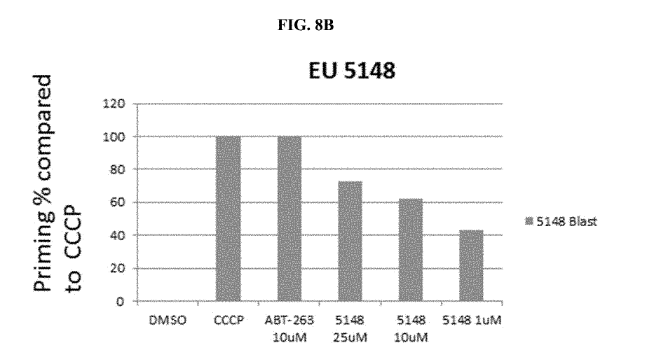

FIG. 8A and FIG. 8B each show a flow cytometry-based assay for detecting MOMP in AML cell line MOLM13 as caused by novel Mcl-1 inhibiting compounds EU5148 (FIG. 8A) and EU5346 (FIG. 8B). As shown in FIG. 8A and FIG. 8B, both compounds induced MOMP, with induction slightly less active that of ABT263 as expected by the relative “priming” by Mcl-1 compared to Bcl-2 and Bcl-xl

### EQUIVALENTS

The detailed description herein describes various aspects and embodiments of the invention, however, unless otherwise specified, none of those are intended to be limiting. Indeed, a person of skill in the art, having read this disclosure, will envision variations, alterations, and adjustments that can be made without departing from the scope and spirit of the invention, all of which should be considered to be part of the invention unless otherwise specified. Applicants thus envision that the invention described herein will be limited only by the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

1. A method for determining a cancer treatment for a patient, comprising:
a) isolating a cancer cell or specimen from said patient;
b) contacting said cancer cell or specimen with one or more therapeutic agents and one or more BH3 domain peptides or mimetics thereof;
c) comparing the level of mitochondrial priming in a test sample with that of the cancer cell or specimen, and determining whether said BH3 domain peptide or mimetic thereof induces apoptosis in said cancer cell to produce a mitochondrial profile for the patient's tumor or cancer cell specimen;
d) determining a correlation between the data obtained from the mitochondrial profile and the sensitivity of said cell or specimen to said treatment; and
e) classifying the patient for likelihood of clinical response to one or more cancer treatments, wherein the mitochondrial profile correlates with treatment efficacy.

2. (canceled)

3. (canceled)

4. The method of claim 1, wherein apoptosis induction is measured through changes in a marker.

5. The method of claim 4, wherein the marker is a change in mitochondrial membrane potential or cytochrome C release.

6. The method of claim 1, wherein the therapeutic agent is contacted with the cell or specimen in vitro.

7. The method of claim 1, wherein the therapeutic agent is contacted with the cell or specimen in vivo.

8. The method of claim 1, wherein the cancer is a hematologic cancer.

9. The method of claim 8, wherein the hematologic cancer is selected from acute myelogenous leukemia (AML), multiple myeloma, follicular lymphoma, acute lymphoblastic leukemia (ALL), chronic lymphocytic leukemia, and non-Hodgkin's lymphoma.

10. The method of claim 1, wherein the cancer is dependent on BH3 containing polypeptides for survival.

11. The method of claim 10, wherein the cancer is dependent on Bcl-2 family polypeptides for survival.

12. The method of claim 1, wherein the cancer treatment is one or more of anti-cancer drugs, chemotherapy, antagonist of an anti-apoptotic protein, surgery, adjuvant therapy, and neoadjuvant therapy.

13. The method of claim 12, wherein the cancer treatment is one or more of a BH3 mimetic, proteasome inhibitor, histone deacetylase inhibitor, glucocorticoid, steroid, monoclonal antibody, antibody-drug conjugate, or thalidomide derivative.

14. The method of claim 12, wherein the cancer treatment is a BH3 mimetic.

15. The method of claim 14, wherein the BH3 mimetic is selected from the group consisting of EU-5148, ABT-263, and EU-5346.

16. The method of claim 12, wherein the cancer treatment is an inhibitor of Bcl-2.

17. The method of claim 12, wherein the cancer treatment is an inhibitor of Mcl-1.

18. The method of claim 1, wherein the mitochondrial profiling further comprises
a) permeabilizing the patient's cancer cells;
b) determining a change in mitochondrial membrane potential upon contacting the permeabilized cells with the one or more therapeutics and the one or more BH3 domain peptides or mimetics thereof; and
c) correlating a loss of mitochondrial membrane potential with chemosensitivity of the cells to apoptosis-inducing chemotherapeutic agents.

19. The method of claim 1, wherein the mitochondrial profiling comprises the use of one or more peptides selected from the group consisting of BIM, BIM2A, BAD, BID, HRK, PUMA, NOXA, BMF, BIK, and PUMA2A.

20. The method of claim 1, wherein said one or more BH3 domains peptides are selected from the group consisting of SEQ ID NOs: 1-14.

21. The method of claim 19, wherein the peptide is used at a concentration of 0.1 μM to 200 μM.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. The method of claim 1, wherein the likelihood of clinical response is defined by the following equation:
$$\% Priming = [ 100 * ( \frac{DMSO AUC - {Peptide}_{1} AUC}{DMSO AUC - {CCCP}_{avg} AUC} ) ] {Peptide}_{1} + [ 100 * ( \frac{DMSO AUC - {Peptide}_{2} AUC}{DMSO AUC - {CCCP}_{avg} AUC} ) ] {Peptide}_{2} + ⋯ / ( n peptides )$$
wherein:
the AUC comprises either area under the curve or signal intensity;
the DMSO comprises the baseline negative control; and
the CCCP (Carbonyl cyanide m-chlorophenyl hydrazone) comprises an effector of protein synthesis by serving as uncoupling agent of the proton gradient established during the normal activity of electron carriers in the electron transport chain in the mitochondria comprises the baseline positive control.

34. (canceled)

35. (canceled)

36. (canceled)

37. (canceled)

38. (canceled)

39. (canceled)

40. (canceled)


SEQUENCE LISTING (14 sequences)

SEQ ID NO: 1 - protein, 20 aa, Homo sapiens
EDIIRNIARHLAQVGDSMDR

SEQ ID NO: 2 - protein, 20 aa, Homo sapiens
MRPEIWIAQELRRIGDEFNA

SEQ ID NO: 3 - protein, 20 aa, Homo sapiens [sequence omitted]

SEQ ID NO: 4 - protein, 24 aa, Homo sapiens
NLWAAQRYGRELRRMSDEFVDSFK

SEQ ID NO: 5 - protein, 20 aa, Homo sapiens
MEGSDALALRLACIGDEMDV

SEQ ID NO: 6 - protein, 20 aa, Homo sapiens
AELPPEFAAQLRKIGDKVYC

SEQ ID NO: 7 - protein, 20 aa, Homo sapiens
PADLKDECAQLRRIGDKVNL

SEQ ID NO: 8 - protein, 20 aa, Homo sapiens
SSAAQLTAARLKALGDELHQ

SEQ ID NO: 9 - protein, 20 aa, Homo sapiens
EQWAREIGAQLRRMADDLNA

SEQ ID NO: 10 - protein, 20 aa, Homo sapiens
HQAEVQIARKLQLIADQFHR

SEQ ID NO: 11 - protein, 20 aa, Homo sapiens
VVEGEKEVEALKKSADWVSD

SEQ ID NO: 12 - protein, 20 aa, Homo sapiens
HQAEVQIARKLQLIADQFHR

SEQ ID NO: 13 - protein, 25 aa, Homo sapiens
NLWAAQRYGRELRRMSDEFVDSFKK

SEQ ID NO: 14 - protein, 25 aa, Homo sapiens
LWAAQRYGREARRMSDEFEGSFKGL